(19) United States
(12) Patent Application Publication
(10) Pub. No.: US 2007/0190004 A1
(43) Pub. Date: Aug. 16, 2007

(54) Substances with a probiotic action used in deodorants

(76) Inventors: Dirk Bockmuhl, Wuppertal (DE); Heide-Marie Hohne, Elsdorf (DE); Claudia Jassoy, Dusseldorf (DE); Regine Scholtyssek, Mettmann (DE); Bernhard Banowski, Dusseldorf (DE); Armin Wadle, Erkrath (DE); Andrea Sattler, Dusseldorf (DE); Roland Breves, Mettmann (DE); Sllke Nieveler, Monchengladbach (DE)

Correspondence Address: PAUL & PAUL, 2000 MARKET STREET, PHILADELPHIA, PA, 19103-3229, US

(21) Appl. No.: 11/522,766

(22) Filed: Sep. 18, 2006

Related U.S. Application Data
(63) Continuation of application No. PCT/EP05/02606, filed on Mar. 11, 2005.

(30) Foreign Application Priority Data
Mar. 18, 2004 (DE) 10 2004 013 694.7
Jul. 7, 2004 (DE) 10 2004 032 734.3

(51) Int. Cl. A61K 36/87; A61K 36/82; A61K 36/328; A61K 36/23; A61K 36/61

(52) U.S. Cl. 424/65; 435/32; 424/729; 424/725; 424/748; 424/769; 424/773

(57) ABSTRACT

A method for promoting probiotic activity on the skin comprises contacting the skin with a probiotic effective amount of a substance having a probiotic effect selected from the group consisting of a plant extract, a glycerol monoalkyl ether, and a fatty acid ester or a combination of the plant extract and the glycerol monoalkyl ether whereby the growth of desired skin microbes is promoted and the growth of undesired skin microbes is inhibited.

### CROSS-REFERENCE TO RELATED APPLICATIONS

This application is a continuation under 35 U.S.C. §§ 365(c) and 120 of International Application No. PCT/EP2005/002606, filed Mar. 11, 2005. This application also claims priority under 35 U.S.C. § 119 of German Application Nos. DE 10 2004 013 694.7, filed Mar. 18, 2004, and DE 10 2004 032 734.3, filed Jul. 7, 2004.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention.

The present invention relates to substances, in particular, plant extracts, which have a probiotic action on the skin, especially in the axillary area, to topical cosmetic and pharmaceutical compositions comprising these substances, and to the use of these substances and compositions, in particular, for combating body odor.

Body odor arises as a result of the degradation of sweat constituents by bacteria in skin flora. For this reason, antibacterial substances have been used in deodorants for a long time. However, the use of unselectively antibacterially effective substances has the disadvantage that bacteria which cause no odor are inhibited or killed. The protective function for the skin which emanates from these bacteria is thus destroyed through the use of unselectively antibacterial active ingredients. In addition, it is known that many antibacterial active ingredients have poor effectiveness against body odor. It can be concluded from this that through the antibacterial active ingredients used, the odor-forming bacteria are not or are not sufficiently inhibited and that interrelations in bacterial symbiosis may exist (e.g., hitherto unknown odor-producing types) whose effects on the formation of body odor are not dealt with by the substances used.

There is, therefore, a need to have available compositions for combating body odor, in particular, for the axillary area, in particular, those which are more effective against body odor than the compositions known to date. These compositions should also advantageously have a selective action against the odor-forming microorganisms.

It was here preferably an object of the present invention to provide compositions which, on the skin, promote the growth and/or the physiological activity of desirable microbes in skin flora compared to the growth and/or the physiological activity of undesired microbes in skin flora. Such substances are also referred to as “probiotic.”

In particular, it was an object of the present invention here to discover substances which promote the growth and/or the physiological activity of odor-neutral microbes and/or inhibit the growth and/or the physiological activity of odor-forming and/or unpleasant odor-forming microbes and, in so doing, be able to bring about a probiotic effect particularly in the axillary area.

For the axillary area, no substances with a probiotic action have hitherto been described, in particular, no substances which selectively promote the growth and/or the physiological activity of odor-neutral microbes at the expense of the growth and/or the physiological activity of odor-forming and/or of unpleasant odor-causing microbes.

(2) Description of Related Art, Including Information Disclosed Under 37 C.F.R. §§ 1.97 and 1.98

The demonstration of a probiotic effect of substances has hitherto essentially been restricted to the intestine. Thus, various publications describe the use of substances which promote the growth of desirable intestinal bacteria. Ahn et al., (1990) Microbial Ecology in Health and Disease 3, 223-229, describes here in particular, also the use of a ginseng extract.

For the skin it has hitherto merely been reported that an oligosaccharide brings about a probiotic effect (advertising brochure for BioEcolia® from Solabia Group, France) in that it can preferably be utilized selectively by saprophytic bacteria. In this regard, it has been shown that the oligosaccharide used promotes the growth of Micrococcus kristinae both compared to the growth of Staphylococcus aureus and also compared to the growth of Corynebacterium xerosis.

Furthermore, it has been reported in EP 1050300 that a mixture of farnesol and xylitol can be used as probiotic substance since this mixture has a selective antibacterial action toward S. aureus and can thus better increase the competing microbe S. epidermidis.

The antibacterial and deodorizing active ingredients are extracts from citrus fruit seeds known in the prior art from the documents EP 911019 and JP 9040516, although no probiotic effect has been described for these.

### BRIEF SUMMARY OF THE INVENTION.

As a prerequisite to achieving the object according to the invention, investigations were conducted to ascertain the differences in the microflora profile between subjects, especially male, with a strong and weak odor in order, in so doing, to discover microbes which are responsible for the formation of odor. In this connection, it has been established that subjects with a strong and/or unpleasant body odor have a different bacterial microflora composition than subjects with a weak body odor.

According to the invention, through the use of biomolecular methods it was possible to discover the following characteristics of people with a strong body odor as typical:
a) reduced fraction of Staphylococcus epidermidis
b) increased fraction of Staphylococcus hominis
c) slightly increased fraction of Anaerococcus octavius
d) slightly increased fraction of certain Corynebacterium species

In particular, the ratio between the two Staphylococcus species appears to be of particular importance here. A possible significance of the Staphylococci ratio on the body odor has hitherto not been described in the prior art; a possible significance ofAnaerococci for body odor likewise as little. By contrast, the prior art has already described howCorynebacteria andmicrococci could be involved in odor formation.

On the basis of the results according to the invention, a search has now been made for substances which shift the microflora profile of subjects with a strong odor or of subjects with an unpleasant body odor in the direction of the microflora profile of subjects with a weak odor, in particular, by selectively promoting the growth of odor-neutral microorganisms, in particular, odor-neutral Staphylococci, primarily of S. epidermidis, and at the same time preventing the growth of odor-forming Staphylococci, in particular, of S. hominis, and/or of Gram-positive anaerobic cocci, in particular, of Streptacocci, primarily of Anaerococcus octavius, and/or of odor-forming Corynbacteria and/or of odor-forming micrococci, primarily of Micrococcus luteus. The minimum requirement for such probiotics was the inhibition of the odor-forming microorganisms without directly influencing the odor-neutral ones, or the promotion of the odor-neutral microorganisms without directly influencing the odor-forming ones.

Surprisingly, substances have now been found which bring about a probiotic effect on the skin by promoting the growth and/or the physiological activity of S. epidermidis and at the same time inhibiting the growth and/or the physiological activity of S. hominis or at least not promoting S. hominis in its growth.

In one preferred embodiment, the plant extracts are as follows:
1. seed extract from grapes (Vitis viticola) (Cosmetochem; water/propylene glycol extract)
2. leaf extract from white tea (Camellia sinensis), (Cosmetochem; water/ethanol dry extract)
3. extract from carcade (Hibiscus, Sudanese tea, Hibiscus sabdariffa), (Cosmetochem; water/ethanol dry extract)
4. flower extract from mallow (Malva sylvestris), (Cosmetochem; water/ethanol dry extract)
5. extract from wine grapes (Vitis viticola), (Cosmetochem; water/propylene glycol extract)
6. mixed extract of carrot and jojoba (Daucus carota and Simmondsia chinensis), (Flavex, CO2 extract)
7. extract from myrrh (Commiphora myrrh), (Cosmetochem; water/propylene glycol extract)
8. extract from marigold (Calendula officinalis), (Cosmetochem; water/ethanol dry extract)

In further preferred embodiments, the substances with a probiotic effect are glycerol monoalkyl ethers and/or esters of organic acids.

The present invention, therefore, first provides a method for identifying substances with a probiotic action in the axillary area, comprising the following steps:
a) taking of comparison samples from subjects with a strong or weak odor,
b) identifying microbes which arise to a greater or lesser degree in subjects with a strong odor compared to subjects with a weak odor, through analysis of the compositions of the comparison samples by biomolecular methods,
c) scanning a substance library for substances which inhibit the growth of the microbes which occur to an increased degree according to (b) and/or promote the growth of the microbes which occur to a lesser degree according to (b).

The present invention, therefore, further provides a method for identifying substances with a probiotic action on the skin, especially in the axillary area, characterized in that a search is made for substances which shift the microflora profile of subjects with a strong odor in the direction of the microflora profile of subjects with a weak odor and/or that a search is made for substances which selectively promote the growth and/or the physiological activity of odor-neutral microorganisms, in particular, odor-neutral Staphylococci, primarily of S. epidermidis, and/or at the same time inhibit the growth and/or the physiological activity of odor-forming Staphylococci, in particular, of S. hominis, and/or of Gram-positive anaerobic cocci, in particular, of Streptococci, primarily of Anaerococcus octavius, and/or of odor-formingCorynebacteria and/or of odor-forming micrococci, primarily of Micrococcus luteus. The search for substances can be carried out here, for example, by screening a substance library. The probiotics here are at least inhibitory to the growth and/or the physiological activity of the odor-forming microorganisms without directly influencing the odor-neutral microorganisms, or they at least promote the growth and/or the physiological activity of the odor-neutral microorganisms without having a direct influence on the odor-forming microorganisms. In a particularly preferred embodiment, a search is made for microorganisms which inhibit the growth and/or the physiological activity of S. hominis and at the same time have no influence on or promote the growth and/or the physiological activity of S. epidermidis.

The screening methods which can be used are Fluorescence in situ Hybridization (FISH) (Amann et al. (1990) J. Bacteriol. 172, 762), Denaturing Gradient Gel Electrophoresis (DGGE) (Fisher and Lerman (1979) Cell 16, 191-200; Myers et al. (1987) Methods in Enzymology 212, 71-104; Electrophoresis (1989) 10(5-6)377-89; Muyzer et al. (1996) Molecular Microbial Ecology Manual 3.4.4:1-23: Eds: Akkermans et al., Holland 1996; Diez et al. (2001) Appl. Environ. Microbiol. 67(7), 2942-2951), Temperature Gradient Gel Electrophoresis (TGGE) (Riesner, D. et al. (1989) Electrophoresis 10 (377-389), Real-Time PCR (Genome Res. (1996) 6(10) 986-94), DNA-Arrays (Applied and Environmental Microbiology (2001) 67(8) 3677-82), SAGE (Velculescu, V.E. et al. (1995) (Serial analysis of gene expression. Science, 270, 484-487) and DNA sequencing techniques (Olsen, G. 1988. Phylogenetic analysis using ribosomal RNA. Method Enzymol. 164:793-812).

### BRIEF DESCRIPTION OF THE SEVERAL VIEWS OF THE DRAWING(S)

Not Applicable

### DETAILED DESCRIPTION OF THE INVENTION

According to the invention, the Gram-positive anaerobic cocci are preferably bacteria of the genus Peptostreptococcus. The generic name Peptostreptococcus includes the genera synonyms Peptoniphilus, Gallicola, Slackia, Anaerococcus (including Anaerococcus octavius), Finegoldia, Micromonas, Atopobium and Ruminococcus. The Gram-positive anaerobic cocci involved in the formation of body odor against which the substances according to the invention are effective are, therefore, chosen from bacteria of these genera in a preferred embodiment.

According to the invention, probiotic action is understood as meaning that the growth and/or the physiological activity of the desired, in particular, skin-friendly and/or odor-neutral, skin microbes or microflora is promoted compared to the growth and/or the ability to survive of the undesired, in particular, skin-unfriendly and/or odor-forming, skin microbes or microflora. This can either be achieved by the active ingredient promoting the growth of the desired skin microbes without directly influencing the growth of the undesired skin microbes, or by the active ingredient inhibiting the growth of the undesired skin microbes without directly influencing the growth of the desired skin microbes. In one embodiment which is particularly preferred and particularly surprising according to the invention, however, the active ingredient promotes the growth of the desired skin microbes and at the same time inhibits the growth of the undesired skin microbes.

The undesired microbes here may in particular, be skin-unfriendly and/or pathogenic microbes and/or microbes which have an excessively high microbial density compared to the occurrence in a healthy person and, therefore, in some instances, bringing about an undesired and/or pathogenic effect. However, the undesired microbes may also be odor-forming microbes or microbes which cause unpleasant odor.

The desired microflora here can, accordingly, in particular, be skin-friendly and/or nonpathogenic microbes, specifically of the resident skin flora, saprophytic microbes or else, in the case, for example, of body odor, microbes which are odor-neutral, i.e. produce no foul-smelling compounds from sweat constituents or other substances. In particular, it should be taken into consideration here that by promoting the growth of the desired microbes, the undesired microbes are suppressed, and vice versa by inhibiting the growth of the undesired microbes, the growth of the desired microbes is promoted, meaning that the probiotic effect can be caused in various ways.

In the case of (unpleasant) body odor, and in particular, axillary odor, the undesired microbes are not necessarily pathogenic microbes, but the odor-forming microbes may naturally likewise be in themselves skin-friendly microbes. In the case of (unpleasant) body odor, the undesired microbes are thus defined as causing body odor. In this connection, a probiotic substance is notable for the fact that it promotes the growth of odor-neutral microbes at the expense of the growth of the odor-forming (unpleasant body odor causing) microbes.

According to the invention “odor-forming microbes” or “odor microbes” are in principle understood as meaning those microorganisms which occur to an increased degree in people with body odor. These are preferably microorganisms which either themselves produce substances or promote the formation of substances which cause an unpleasant odor. Furthermore, these may be microorganisms which are involved only directly in the formation of such substances, for example, by producing a substance or promoting the formation of substances which can be converted by other microorganisms to unpleasantly smelling substances. According to the invention, the odor-forming microorganisms do not necessarily have to cause the unpleasant odor themselves, but can also be involved in the metabolism of odor formation in another way.

The present invention further provides the use of substances with a probiotic action, especially with a probiotic action on the skin, in particular, in the axillary area, in particular, plant extracts, glycerol monoalkyl ethers or esters of organic acids for promoting the growth and/or the physiological activity of desired skin microbes, where the desired skin microbes are preferably benign and/or nonpathogenic and/or skin-friendly and/or saprophytic skin microbes and/or, and particularly preferably, odor-neutral microbes, in particular, odor-neutral coagulase-negative Staphylococci, here especially S. epidermidis.

The present invention thus further provides the use of substances with a probiotic action, in particular, with a probiotic acid on the skin, in particular, in the axillary area, in particular, plant extracts, glycerol monoalkyl ethers or esters of organic acids for inhibiting growth and/or the physiological activity of undesired skin microbes, where the undesired microbes are preferably skin-unfriendly microbes and/or pathogenic microbes and/or, and particularly preferably, odor-forming microbes, in particular, odor-forming Staphylococci, primarily S. hominis, odor-forming Gram-positive anaerobic cocci, in particular, Peptostreptococci, especially Anaerococcus octavius, and/or odor-forming corynebacteria and/or odor-forming micrococci, especially Micrococcus luteus.

The use can take place here in particular, in each case in cosmetic topical skin-treatment compositions, the composition of which is further specified in this description.

According to the invention, the term “skin” is preferably the skin itself, in particular, the human skin, but in addition also the mucosa, and skin appendages if they contain living cells, in particular, hair follicle, hair root, hair bulb, the ventral epithelium of the nail bed (Lectulus), and sebaceous glands and sweat glands. In one preferred embodiment, skin is understood according to the invention as meaning the skin in the armpit area (axillary area).

Preferably, the substance with a probiotic action on the skin according to the invention is suitable for shifting the microflora profile which occurs in people with a strong and/or unpleasant body odor toward the microflora profile which arises in people without body odor, or is able to restore and/or to stabilize such a microflora profile.

The present invention, therefore, further provides a probiotic substance which has an odor-inhibiting action in the armpit area, preferably by promoting growth of odor-neutral Staphylococci, in particular, S. epidermidis, and/or inhibiting growth of odor-forming Staphylococci, in particular, S. hominis, and/or inhibiting growth of Gram-positive anaerobic cocci, in particular, Anaerococcus octavius and/or inhibiting growth of odor-forming corynebacteria and/or of odor-forming mirococci, in particular, Micrococcus luteus.

In one embodiment which is particularly preferred according to the invention, the substance with a probiotic action is a plant extract, a glycerol monoalkyl ether or an ester of an organic acid which promotes the growth of odor-neutral coagulase-negative Staphylococci, in particular, of S. epidermidis, and at the same time exhibits an inhibiting or no direct effect on the growth of odor-forming Staphylococci, in particular, S. hominis.

In a further embodiment which is particularly preferred according to the invention, the substance with a probiotic action is a plant extract, a glycerol monoalkyl ether or an ester of an organic acid which has an inhibiting effect on the growth of odor-forming Staphylococci, in particular, S. hominis, and at the same time exhibits a promoting or no direct effect on the growth of odor-neutral Staphylococci, in particular, S. epidermidis.

The present invention further provides the use of the substances with a probiotic action, and in particular, with a probiotic action on the skin, in cosmetic topical skin-treatment compositions for treating body odor, in particular, in the armpit area, primarily through use in deodorants and/or antiperspirants.

The present invention further provides the use of the substances with a probiotic action, and in particular, with a probiotic action on the skin, in cosmetic topical skin-treatment compositions for treating blemished, dry or greasy skin, and for treating skin fungi or dandruff.

The treatment can take place here in each case also preventatively or prophylactically.

The present invention further provides a cosmetic or pharmaceutical composition comprising a substance with a probiotic action on the skin, especially a plant extract with a probiotic action on the skin, a glycerol monoalkyl ether with a probiotic action on the skin, an ester of an organic acid with a probiotic action on the skin, or mixtures thereof, where the cosmetic or pharmaceutical composition is preferably a topical skin-treatment composition, in particular, a deodorant and/or antiperspirant.

The substance with a probiotic action is present in the composition here preferably in an amount of from 0.01 to 20% by weight, particularly preferably from 0.05 to 10% by weight, especially from 0.1 to 5% by weight, in particular, from 0.1 to 1.5% by weight or from 0.5 to 2% by weight, based on the total weight of the composition.

The plant extract with a probiotic action according to the invention is preferably a tea extract, in particular, from the Theaceae family or from the Malvaceae family, is an extract from the Vitaceae family, from the Apiaceae family, the Buxaceae or is an extract from the Asteraceae family or mixtures thereof. Particular preference is given to an extract from the Vitaceae family.

The extract from the Theaceae family is preferably an extract from Camellia spec., especially an extract from white tea (Camellia sinensis). In one preferred embodiment, it is an extract from the leaves, as is obtainable, for example, from Cosmetochem.

The extract from the Malvaceae family is preferably an extract from Hibiscus spec., especially an extract from Sudanese tea (carcade, hibiscus, Hibiscus sabdariffa), or an extract from Malva spec., especially an extract from mallow (Malva sylvestris), in particular, mallow blossom.

The extract from the Vitaceae family is preferably an extract from Vitis spec., in particular, an extract from the grape (Vitis viticola). Here, it is particularly preferably an extract from grape seeds.

The extract from the Apiaceae family is preferably an extract from Daucus spec., especially from carrot (Daucus carota), or an extract from Commiphora spec., especially from myrrh (Commiphora myrrh). In one preferred embodiment, this is an extract from the roots, as is obtainable, for example, from Cosmetochem or Rahn.

The extract from the Buxaceae family is preferably an extract from Simmondsia spec., especially from jojoba (Simmondsia chinensis).

The extract from the Asteraceae family is preferably an extract from Calendula spec., especially from marigold (Calendula officinalis).

The above-mentioned plant extracts are obtainable, for example, from the companies Cosmetochem (Germany) or Rahn (Germany).

The plant extract with a probiotic action can in principle be prepared in any manner known to the person skilled in the art using any desired plant tissue and using any desired extractant. First, the plant extract can be carried out, for example, by extraction of the whole plant, by extraction from flowers, leaves, seeds, roots and/or by extraction from the meristem of the plant.

The extractants used for producing said plant extracts may, for example, be water, alcohols, and mixtures thereof. Suitable alcohols are, for example, lower alcohols, such as ethanol and isopropanol, but in particular, also polyhydric alcohols, such as ethylene glycol, propylene glycol and butylene glycol, either as sole extractant, or in a mixture with water. Thus, for example, plant extracts based on water/propylene glycol in the ratio 1:10 to 10:1 have proven to be particularly suitable. The extraction can be carried out, for example, in the form of steam distillation. In some instances, a dry extraction can also be carried out.

Following the extraction, the extracts can also be applied to supports, in particular, in order to be able to be better incorporated into products. Supports suitable according to the invention are, for example, maltodextrin and talc. In one preferred embodiment of the present invention, the choice of extract is governed by the preparation in which the extract is to be used. For example, aqueous and alcoholic extracts, in particular, water/propylene glycol extracts, are preferably used in aqueous or alcoholic preparations or soap-containing sticks, oil-soluble extracts are preferably used in oil-containing preparations, in particular, in antiperspirant sticks or antiperspirant aerosols, extracts on maltodextrin supports can be used either in hydrophilic or in hydrophobic products, extracts on talc supports are preferably used in hydrophobic products.

Accordingly, the present invention also provides, in particular, cosmetic or pharmaceutical compositions which comprise the plant extracts with a probiotic action according to the invention, in particular, at least one of those mentioned above, on supports, in particular, on talc supports or on maltodextrin supports, the use of which has proven particularly advantageous according to the invention.

The extract from Theaceae, Malvaceae and Asteraceae is preferably a water/propylene glycol extract or a water/ethanol dry extract or a water/ethanol extract on maltodextrin or on talc supports, the extract from Vitaceae is preferably a water/propylene glycol extract, the extract from Apiaceae is preferably a CO₂ extract or a water/propylene glycol extract and the extract from Buxaceae is preferably a CO₂ extract.

The plant extracts with a probiotic action on the skin can be used according to the invention either in pure form or in dilute form. If they are used in dilute form, they usually comprise about 2-80% by weight of active substance and, as solvent, the extractant or extractant mixture used for their isolation. Depending on the choice of extractant, it may be preferred to stabilize the plant extract by adding a solubility promoter. Suitable solubility promoters are, for example, ethoxylation products of optionally hydrogenated vegetable and animal oils. Preferred solubility promoters are ethoxylated mono-, di- and triglycerides of C₈₋₂₂ fatty acids with 4 to 50 ethylene oxide units, e.g., hydrogenated ethoxylated castor oil, olive oil ethoxylate, almond oil ethoxylate, mink oil ethoxylate, polyoxyethylene glycol caprylic/capric glycerides, polyoxyethylene glycerol monolaurate and polyoxyethylene glycol coconut fatty acid glycerides.

The glycerol monoalkyl ether with a probiotic action is preferably a 1-alkyl glycerol ether. The alkyl radical here is preferably a (C₂-C₄)-, in particular, a (C₄-C₁₂)-, especially a (C₆-C₁₀)-alkyl radical, where the alkyl radical may either be straight-chain or branched. In one particularly preferred embodiment, the alkyl radical is a branched octyl radical and/or an alkylhexyl radical, in particular, an ethylhexyl radical, especially a 2-ethylhexyl radical. 1-(2-Ethylhexyl) glycerol ether is obtainable, for example, under the trade name Sensiva® SC 50 (Schülke & Mayr, Germany).

The ester of an organic acid with a probiotic action is preferably an ester of a (C₁₀-C₁₈)-carboxylic acid with a (C₁-C₁₀)-alcohol, where either the carboxylic acid radical or the alcohol radical may be linear or branched and saturated or unsaturated, and where the alkyl groups of the carboxylic acid radical and of the alcohol radical can, independently of one another, carry one or more substituents, in particular, chosen from (C₁-C₆)-alkyl and hydroxy. The carboxylic acid is particularly preferably a (C₁₂-C₁₆)-carboxylic acid, especially a C₁₄-carboxylic acid, in particular, myristic acid. The alcohol is particularly preferably a (C₁-C₆)-alkanol, especially methanol, ethanol, propanol, in particular, 1-propanol, 2-propanol or isopropanol, butanol, in particular, 1-butanol, 2-butanol or tert-butanol, pentanol, in particular, 1-pentanol, 2-pentanol, 3-pentanol, 2-methyl-1-butanol, 3-methyl-1-butanol, 2-methyl-2-butanol or 3-methyl-2-butanol. In a particularly preferred embodiment, the ester with a probiotic action is isopropyl myristate or ethyl myristate.

The cosmetic or pharmaceutical composition according to the invention may be any desired administration form, for example, a solid or liquid soap, a lotion, a spray, a cream, a gel, an emulsion, a cleansing liquid or cleansing milk, a deodorant, an antiperspirant, an ointment, a hair treatment or a shampoo or it may also be present in any of the described or other administration forms, for example, also in a plaster, in particular, in a gel reservoir plaster or matrix plaster.

In one embodiment which is particularly preferred according to the invention, the cosmetic or pharmaceutical composition is a deodorant and/or antiperspirant. The deodorant and/or antiperspirant here is preferably in the form of a powder, in stick form, in the form of a syndet, washing lotion, aerosol spray, pump spray, liquid or gel-like roll-on application, cream, foam, liquid or solid soap, gel or in the form of an impregnated flexible substrate.

Accordingly, applicators which can be used are, depending on the application form, for example, stick holder, roll-on, pump, tube, small pot, dispenser, wipe, aerosol can or bottle.

A suitable application site is the skin in any area of the body, in particular, the facial skin, the scalp, the skin on the feet and hands. In one particularly preferred embodiment, the site of application is the skin in the axillary area.

The cosmetic or pharmaceutical composition according to the invention can also comprise constituents other than those specified above. In one preferred embodiment, it comprises at least one of the substances listed below. It can also comprise any desired combination of the constituents listed below.

In particular, cosmetic or pharmaceutical compositions which have proven particularly advantageous according to the invention are those which comprise mixtures of at least one plant extract with a probiotic action on the skin according to the invention and at least one glycerol monoalkyl ether with a probiotic action on the skin.

In addition, cosmetic or pharmaceutical compositions which have proven particularly advantageous according to the invention are those which comprise mixtures of at least one substance with a probiotic action on the skin according to the invention and at least one deodorant active ingredient, in particular, a substance with an antimicrobial action. In the case of the combined use of a substance with a probiotic action together with a deodorant active ingredient, a synergistic effect advantageously arises here which consists in the bacterial count being reduced overall, but the undesired bacteria being decimated to a greater degree than the desired bacteria. On account of this combined effect, the desired bacteria are then able, in some instances to an even greater degree, to spread on the skin than would be the case for the existence of a purely probiotic effect.

In one embodiment according to the invention, besides at least one plant extract with a probiotic action according to the invention, the composition comprises at least one further plant extract. This further plant extract can be produced, for example, by extraction of the whole plant but also exclusively by extraction from flowers and/or leaves and/or seeds and/or other plant parts. According to the invention, the extracts from the meristem, i.e. the formation tissue of the plants which is capable of dividing, and the extracts from specific plants such as green tea, hamamelis, chamomile, pansy, peony, aloe vera, horse chestnut, sage, willow bark, cinnamon tree, chrysanthemum, oak bark, stinging nettle, hops, burdock, horsetail, hawthorn, linden blossom, almond, fir needle, sandalwood, juniper, coconut, kiwi, guava, lime, mango, apricot, wheat, melon, orange, grapefruit, avocado, rosemary, birch, beech shoots, lady's smock, yarrow, wild thyme, thyme, melissa, restharrow, marshmallow (Althaea), violet, blackcurrant leaves, coltsfoot, cinquefoil, ginseng, ginger root and sweet potato are preferred as further plant extract. Algae extracts can also advantageously be used. The algae extracts used according to the invention originate from green algae, brown algae, red algae or blue algae (cyanobacteria). The algae used for the extraction may either be of natural origin or obtained by biotechnological processes and, if desired, modified compared to the natural form. The modification of the organisms can take place by genetic engineering, by growing or by cultivation in media enriched with selected nutrients. Preferred algae extracts originate from seaweed, blue algae, from the green algae Codium tomentosum, and from the brown algae Fucus vesiculosus. A particularly preferred algae extract originates from blue algae of the species Spirulina which have been cultivated in a magnesium-enriched medium.

As further plant extract, particular preference is given to the extracts from Spirulina, green tea, aloe vera, meristem, hamamelis, apricot, guava, sweet potato, lime, mango, kiwi, cucumber, mallow, marshmallow and violet. As additional plant extract, the compositions according to the invention can also comprise mixtures of two or more, in particular, of two, different plant extracts.

As for producing the plant extracts with a probiotic action, extractants for producing the specified further plant extracts which can be used are, for example, water, alcohols, and mixtures thereof. Among the alcohols, preference is given here to lower alcohols, such as ethanol and isopropanol, but in particular, polyhydric alcohol such as ethylene glycol, propylene glycol and butylene glycol, either as sole extractant or in a mixture with water. Plant extracts based on water/propylene glycol in the ratio 1:10 to 10:1 have proven to be particularly suitable. According to the invention, steam distillation falls under the preferred extraction methods. In some instances, however, the extraction can also take place in the form of dry extraction.

According to the invention, the plant extracts can be used either in pure form or in dilute form. If they are used in dilute form, they usually comprise about 2-80% by weight of active substance and, as solvent, the extractant or extractant mixture used in their isolation. Depending on the choice of extractant, it may be preferred to stabilize the plant extract by adding a solubility promoter. Suitable solubility promoters are, for example, ethoxylation products of optionally hydrogenated vegetable and animal oils. Preferred solubility promoters are ethoxylated mono-, di- and triglycerides of C₈₋₂₂-fatty acids having 4 to 50 ethylene oxide units, e.g., hydrogenated ethoxylated castor oil, olive oil ethoxylate, almond oil ethoxylate, mink oil ethoxylate, polyoxyethylene glycol caprylic/capric glycerides, polyoxyethylene glycerol monolaurate and polyoxyethylene glycol coconut fatty acid glycerides.

In addition, it may be preferred to use mixtures of two or more, in particular, of two, different plant extracts in addition to the plant extract with a probiotic action in the compositions according to the invention.

With regard to the plant extracts which can be used according to the invention, reference is also made to the extracts which are listed in the table starting on page 44 of the 3rd edition of the introduction to the ingredients declaration of cosmetic compositions, published by the Industrieverband Körperpflege- und Waschmittel e.V. (IKW), Frankfurt.

The cosmetic or pharmaceutical compositions and in particular, the deodorant or antiperspirant compositions preferred according to the invention which comprise the substances with a probiotic action according to the invention may also comprise fatty substances. Fatty substances are understood as meaning fatty acids, fatty alcohols, natural and synthetic cosmetic oil components, and natural and synthetic waxes, which may be present either in solid form or else as a liquid in aqueous or oily dispersion.

Fatty acids which can be used are linear and/or branched, saturated and/or unsaturated C₈₋₃₀-fatty acids. Preference is given to C₁₀₋₂₂-fatty acids. Examples are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid, and technical-grade mixtures thereof. The use of stearic acid is particularly preferred. The fatty acids used may carry one or more hydroxy groups. Preferred examples thereof are the α-hydroxy-C₈-C₁₈-carboxylic acids, and 12-hydroxystearic acid. The use amount here is 0.1-15% by weight, preferably 0.5-10% by weight, particularly preferably 1-5% by weight, in each case based on the total composition.

Fatty alcohols which can be used are saturated, mono- or polyunsaturated, branched or unbranched fatty alcohols having 6-30, preferably 10-22 and very particularly preferably 12-22, carbon atoms. For example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and guerbet alcohols thereof can be used for the purposes of the invention.

Waxes are often used for stick formulations. Natural or synthetic waxes which can be used according to the invention are solid paraffins or isoparaffins, plant waxes such as candelilla wax, carnauba wax, esparto grass wax, Japan wax, cork wax, sugar cane wax, ouricury wax, montan wax, sunflower wax, fruit waxes and animal waxes, such as, for example, beeswaxes and other insect waxes, spermaceti, shellac wax, wool wax and uropygial grease, also mineral waxes, such as, for example, ceresin and ozokerite or the petrochemical waxes, such as, for example, petrolatum, paraffin waxes, microwaxes of polyethylene or polypropylene and polyethylene glycol waxes. It may be advantageous to use hydrogenated waxes. In addition, chemically modified waxes, in particular, the hard waxes, e.g., montan ester waxes, sasol waxes and hydrogenated jojoba waxes, can also be used.

Also suitable are the mono-, di- and triglycerides of saturated and optionally hydroxylated C₁₆₋₃₀-fatty acids, such as, for example, hydrogenated triglyceride fats (hydrogenated palm oil, hydrogenated coconut oil, hydrogenated castor oil), glyceryl monostearate (Cutina® MD), glyceryl tribehenate or glyceryl tri-12-hydroxystearate, also synthetic full esters of fatty acids and glycols (e.g., Syncrowachs®) or polyols with 2-6 carbon atoms, fatty acid monoalkanolamides with a C₁₂₋₂₂-acyl radical and a C₂₋₄-alkanol radical, esters of saturated and/or unsaturated, branched and/or unbranched alkanecarboxylic acids of chain length from 1 to 80 carbon atoms and saturated and/or unsaturated, branched and/or unbranched alcohols of chain length from 1 to 80 carbon atoms, including, for example, synthetic fatty acid-fatty alcohol esters, such as stearyl stearate or cetyl palmitate, esters of aromatic carboxylic acids, dicarboxylic acids and hydroxycarboxylic acids (e.g., 12-hydroxystearic acid) and saturated and/or unsaturated, branched and/or unbranched alcohols of chain length from 1 to 80 carbon atoms, lactides of long-chain hydroxycarboxylic acids and full esters of fatty alcohols and di- and tricarboxylic acids, e.g., dicetyl succinate or dicetyl/stearyl adipate, and mixtures of these substances, if the individual wax components or their mixture are solid at room temperature.

It is particularly preferred to choose the wax components from the group of esters of saturated, unbranched alkane carboxylic acids of chain length from 14 to 44 carbon atoms and saturated, unbranched alcohols of chain length from 14 to 44 carbon atoms if the wax component or the totality of the wax components are solid at room temperature. The wax components can be chosen particularly advantageously from the group of C₁₆₋₃₆-alkyl stearates, C₁₀₋₄₀-alkyl stearates, C₂₋₄₀-alkyl isostearates, C₂₀₋₄₀-dialkyl esters of dimer acids, C₁₈₋₃₈-alkylhydroxystearoyl stearates, C₂₀₋₄₀-alkyl erucates, in addition C₃₀₋₅₀-alkyl beeswax and cetearyl behenate can be used. Silicone waxes, for example, stearyltrmethylsilane/stearyl alcohol are also advantageous in some instances. Particularly preferred wax components are the esters of saturated, monohydric C₂₀-C₆₀-alcohols and saturated C₈-C₃₀-monocarboxylic acids, particularly preferably a C₂₀-C₄₀-alkyl stearate which is obtainable under the name Kesterwachs® K82H from Koster Keunen Inc. The wax or the wax components should be solid at 25° C., but melt in the range from 35-95° C., where a range from 45-85° C. is preferred.

Natural, chemically modified and synthetic waxes can be used on their own or in combination.

The wax components are present in an amount of from 0.1 to 40% by weight, based on the total composition, preferably 1 to 30% by weight and in particular, 5-15% by weight.

The compositions according to the invention can also comprise at least one nonpolar or polar liquid oil, which may be natural or synthetic. The polar oil component can be chosen from vegetable oils, e.g., sunflower oil, olive oil, soybean oil, rapeseed oil, almond oil, jojoba oil and the liquid fractions of coconut oil, and synthetic triglyceride oils, from ester oils, i.e. the esters of C₆₋₃₀-fatty acids with C₂₋₃₀-fatty alcohols, from dicarboxylic acid esters, such as di-n-butyl adipate, di(2-ethylhexyl) adipate and di(2-ethyihexyl) succinate, and diol esters, such as ethylene glycol dioleate and propylene glycol di(2-ethylhexanoate), from symmetrical, asymmetrical or cyclic esters of carbonic acid with fatty alcohols, described for example, in DE-A 197 56 454, glycerol carbonate or dicaprylyl carbonate (Cetiol® CC), from mono, di and tri fatty acid esters of saturated and/or unsaturated linear and/or branched fatty acids with glycerol, from branched alkanols, e.g., Guerbet alcohols with a single branch on carbon atom 2, such as 2-hexyldecanol, 2-octyidodecanol, isotridecanol and isohexadecanol, from alkanediols, e.g., the vicinal diols obtainable from epoxy alkanes having 12-24 carbon atoms by ring opening with water, from ether alcohols, e.g., the monoalkyl ethers of glycerol, of ethylene glycol, of 1,2-propylene glycol or of 1,2-butanediol, from dialkyl ethers having in each case 12-24 carbon atoms, e.g., the alkyl methyl ethers or di-n-alkyl ethers having in each case a total of 12-24 carbon atoms, in particular, di-n-octyl ether (Cetiol®OE ex Cognis), and from addition products of ethylene oxide and/or propylene oxide onto mono- or polyhydric C₃₋₂₀-alkanols such as butanol and glycerol, e.g., PPG-3 myristyl ether (Witconol® APM), PPG-14 butyl ether (Ucon Fluid® AP), PPG-15 stearyl ether (Arlamol® E), PPG-9 butyl ether (Breox® B25) and PPG-10 butanediol (Macol® 57). The nonpolar oil component can be chosen from liquid paraffin oils, isoparaffin oils, e.g., isohexadecane and isoeicosane, from hydrogenated polyalkenes, in particular, poly-1-decenes (commercially available as Nexbase 2004, 2006 or 2008 FG (Fortum, Belgium)), from synthetic hydrocarbons, e.g., 1,3-di(2-ethylhexyl)cyclohexane (Cetiol® S), and from volatile and nonvolatile silicone oils, which may be cyclic, such as, for example, decamethylcyclopentasiloxane and dodecamethylcyclohexasiloxane, or linear, e.g., linear dimethylpolysiloxane, commercially available, for example, under the name Dow Corning 190, 200, 244, 245, 344 or 345 and Baysilon® 350 M.

The compositions according to the invention can also comprise at least one water-soluble alcohol. According to the invention, solubility in water is understood as meaning that at least 5% by weight of the alcohol dissolve at 20° C. to give a clear solution, or else—in the case of long-chain or polymeric alcohols—can be brought into solution by heating the solution to 50° C. to 60° C. Depending on the administration form, monohydric alcohols, such as, for example, ethanol, propanol or isopropanol, are suitable. Water-soluble polyols are also suitable. These include water-soluble diols, triols and higher hydric alcohols, and polyethylene glycols. Among the diols, C₂-C₁₂-diols are suitable, in particular, 1,2-propylene glycol, butylene glycols, such as, for example, 1,2-butylene glycol, 1,3-butylene glycol and 1,4-butylene glycol, hexanediols, such as, for example, 1,6-hexanediol. Also preferably suitable are glycerol and, in particular, diglycerol and triglycerol, 1,2,6-hexanetriol, and the dipropylene glycol and the polyethylene glycols (PEG) PEG-400, PEG-600, PEG-1000, PEG-1550, PEG-3000 and PEG-4000.

The amount of alcohol or of alcohol mixture in the compositions according to the invention is 1-50% by weight or 1-70% by weight and preferably 5-40% by weight or 5-55% by weight, based on the total composition. According to the invention, either one alcohol or a mixture of two or more alcohols can be used.

The compositions according to the invention may be essentially anhydrous, i.e. comprise at most 5% by weight, preferably at most 1% by weight, of water. In hydrous administration forms, the water content is 5-98% by weight, preferably 10-90% by weight and particularly preferably 15-85% by weight, based on the total composition.

The compositions according to the invention can also comprise at least one hydrophilically modified silicone. They permit the formulation of highly transparent compositions, reduce the stickiness and leave behind a fresh feel on the skin. According to the invention, hydrophilically modified silicones are understood as meaning polyorganosiloxanes with hydrophilic constituents which bring about the solubility of the silicones in water. According to the invention, solubility in water is understood as meaning that at least 2% by weight of the silicone modified with hydrophilic groups dissolve in water at 20° C. Corresponding hydrophilic substituents are, for example, hydroxy, polyethylene glycol or polyethylene glycol/polypropylene glycol side chains, and ethoxylated ester side chains. Of preferred suitability according to the invention are hydrophilically modified silicone copolymers, in particular, dimethicone copolyols, which are sold, for example, by Wacker-Chemie under the name Belsil® DMC 6031, Belsil® DMC 6032, Belsil® DMC 6038 or Belsil® DMC 3071 VP or by Dow Corning under the name DC 2501. Of particularly preferred suitability is the use of Belsil® DMC 6038 since it allows the formulation of highly transparent compositions which achieve higher acceptance by the consumer. The hydrophilic silicone derivative used may also be ABIL EM97 from Degussa/Goldschmidt. According to the invention, any mixture of the specified silicones can also be used.

The amount of hydrophilically modified silicone or of the alcohol mixture in the compositions according to the invention is 0.5-10% by weight, preferably 1-8% by weight and particularly preferably 2-6% by weight, based on the total weight of the composition.

The compositions according to the invention can also comprise emulsifiers and/or surfactants. In one particularly preferred embodiment, these were addition products of 10-40 mol of ethylene oxide onto linear or branched fatty alcohols having 16-22 carbon atoms, onto fatty acids having 12-22 carbon atoms, onto fatty acid alkanolamides, onto fatty acid monoglycerides, onto sorbitan fatty acid monoesters, onto fatty acid alkanolamides, onto fatty acid glycerides, e.g., onto hydrogenated castor oil, onto methyl glucoside mono fatty acid esters and mixtures thereof. In principle, however, any other emulsifiers and/or surfactants can be used.

Emulsifiers which can be used according to the invention in this sense are, for example,
addition products of from 4 to 30 mol of ethylene oxide and/or 0 to 5 mol of propylene oxide onto linear or branched C₈-C₂₂-fatty alcohols, onto C₁2-C₂₂-fatty acids and onto C₈-C₁₅-alkylphenols,
C₁₂-C₂₂-fatty acid monoesters and diesters of addition products of from 1 to 30 mol of ethylene oxide onto C₃-C₆-polyols, in particular, onto glycerol,
ethylene oxide and polyglycerol addition products onto methyl glucoside fatty acid esters, fatty acid alkanolamides and fatty acid glucamides,
C₈-C₂₂-alkyl monoglycosides and oligoglycosides and ethoxylated analogs thereof, where degrees of oligomerization of from 1.1 to 5, in particular, 1.2 to 2.0, and glucose as sugar component are preferred,
mixtures of alkyl (oligo)glucosides and fatty alcohols, e.g., the commercially available product Montanov®68,
addition products of from 5 to 60 mol of ethylene oxide onto castor oil and hydrogenated castor oil,
partial esters of polyols having 3-6 carbon atoms with saturated C₈-C₂₂-fatty acids,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are beta-sitosterol, stigmasterol, campesterol and ergosterol. Sterols are also isolated from fungi and yeasts, the so-called mykosterols.
phospholipids, primarily the glucose phospholipids, which are obtained, for example, as lecithins or phosphatidylcholines from, for example, egg yolk or plant seeds (e.g., soya beans),
fatty acid esters of sugars and sugar alcohols, such as sorbitol,
polyglycerols and polyglycerol derivatives, preferably polyglyceryl-2 dipolyhydroxystearate (commercial product Dehymuls® PGPH) and polyglyceryl-3 diisostearate (commercial product Lameform® TGI),
linear and branched C₈-C₃₀-fatty acids and their Na, K, ammonium, Ca, Mg and Zn salts.

The compositions according to the invention comprise the emulsifiers preferably in amounts of from 0.1 to 25% by weight, in particular, 0.5-15% by weight, based on the total composition.

In another, likewise preferred embodiment, at least one ionic emulsifier chosen from anionic, zwitterionic, ampholytic and cationic emulsifiers is present. Preferred anionic emulsifiers are alkyl sulfates, alkyl polyglycol ether sulfates and ether carboxylic acids having 10 to 18 carbon atoms in the alkyl group and up to 12 glycol ether groups in the molecule, sulfosuccinic mono- and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups, monoglyceride sulfates, alkyl and alkenyl ether phosphates, and protein fatty acid condensates. Zwitterionic emulsifiers carry at least one quaternary ammonium group and at least one —COO⁻ or —SO₃⁻ group in the molecule. Particularly suitable zwitterionic emulsifiers are the so-called betaines, such as the N-alkyl-N,N-dimethylammonium glycinates, N-acylaminopropyl-N,N-dimethylammonium glycinates and 2-alkyl-3-carboxymethyl-3-hydroxyethylimidazolines having in each case 8 to 18 carbon atoms in the alkyl or acyl group, and cocoacylaminoethyl hydroxyethylcarboxymethylglycinate.

Apart from a C₈-C₂₄-alkyl or acyl group, ampholytic emulsifiers contain at least one free amino group and at least one —COOH or —SO₃H group in the molecule and can form internal salts. Examples of suitable ampholytic emulsifiers are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 24 carbon atoms in the alkyl group.

The ionic emulsifiers are present in an amount of from 0.01 to 5% by weight, preferably from 0.05 to 3% by weight and particularly preferably from 0.1 to 1% by weight, based on the total composition.

Nonionic surfactants which can be used according to the invention are, for example:
alkoxylated fatty acid alkyl esters of the formula R¹CO—(OCH₂CHR²)ₓOR³, in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and x is numbers from 1 to 20,
addition products of ethylene oxide onto fatty acid alkanolamides and fatty amines,
fatty acid N-alkylglucamides,
C₈-C₂₂-alkylamine N-oxides,
alkyl polyglycosides corresponding to the general formula RO—(Z)ₓ where R is a C₈-C₁₆-alkyl group, Z is sugars, and x is the number of sugar units. The alkyl polyglycosides which can be used according to the invention can contain just one specific alkyl radical R. However, these compounds are usually prepared starting from natural fats and oils or mineral oils. In this case, the alkyl radicals R present are mixtures corresponding to the starting compounds or corresponding to the particular work-up of these compounds. Particular preference is given to those alkyl polyglycosides in which R consists essentially of C₈- and C₁₀-alkyl groups, essentially of C₁₂- and C₁₄-alkyl groups, essentially of C₈- to C₁₆-alkyl groups or essentially of C₁₂- to C₁₆-alkyl groups.

Any mono- or oligosaccharides can be used as sugar building block Z. Usually, sugars having 5 or 6 carbon atoms, and the corresponding oligosaccharides are used, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred. The alkyl polyglycosides which can be used according to the invention contain, on average, 1.1 to 5, preferably 1.1 to 2.0, particularly preferably 1.1 to 1.8, sugar units. The alkoxylated homologs of said alkyl polyglycosides can also be used according to the invention. These homologs can comprise on average up to 10 ethylene oxide and/or propylene oxide units per alkyl glycoside unit.

Suitable zwitterionic surfactants are surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example, cocoalkyldimethylammonium glycinate, N-acylaminopropyl-N,N-dimethylammonium glycinates, for example,cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl-hydroxyethylcarboxymethylglycinate. A preferred zwitterionic surfactant is the fatty acid amide derivative known under the INCl name Cocamidopropyl Betaine.

Suitable anionic surfactants in compositions according to the invention are all anionic surface-active substances suitable for use on the human body. These are characterized by a solubilizing, anionic group, such as, for example, a carboxylate, sulfate, sulfonate or phosphate group and a lipophilic alkyl group having about 8 to 30 carbon atoms. In addition, glycol or polyglycol ether groups, ester groups, ether groups and amide groups and also hydroxyl groups may be present in the molecule. Examples of suitable foaming anionic surfactants are, in each case in the form of the sodium, potassium and ammonium, and the mono-, di- and trialkanolammonium salts having 2 to 4 carbon atoms in the alkanol group,
acyl glutamates of the formula (II), 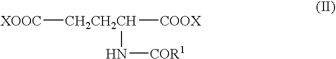
in which R¹CO is a linear or branched acyl radical having 6 to 22 carbon atoms and 0, 1, 2 or 3 double bonds and X is hydrogen, an alkali metal and/or alkaline earth metal, ammonium, alkylammonium, alkanolammonium or glucammonium, for example,acyl glutamates which are derived from fatty acids having 6 to 22, preferably 12 to 18, carbon atoms, such as, for example, C_{12/14}- or C_{12/18}-coconut fatty acid, lauric acid, myristic acid, palmitic acid and/or stearic acid, in particular, sodium N-cocoyl- and sodium N-stearoyl-L-glutamate,
esters of a hydroxy-substituted di- or tricarboxylic acid of the general formula (III), 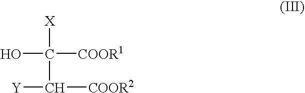
in which X═H or a —CH₂COOR group, Y═H or —OH with the proviso that Y═H if X═—CH₂COOR, R, R¹ and R², independently of one another, are a hydrogen atom, an alkali metal or alkaline earth metal cation, an ammonium group, the cation of an ammonium-organic base or a radical Z which originates from a polyhydroxylated organic compound which are chosen from the group of etherified (C₆-C₁₈)-alkylpolysaccharides having from 1 to 6 monomeric saccharide units and/or etherified aliphatic (C₆-C₁₆)-hydroxyalkylpolyols having 2 to 16 hydroxyl radicals, with the proviso that at least one of the groups R, R¹ or R² is a radical Z,
esters of the sulfosuccinic acid salt of the general formula (IV), 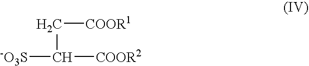
in which R¹ and R², independently of one another, are a hydrogen atom, an alkali metal or alkaline earth metal cation, an ammonium group, the cation of an ammonium-organic base or a radical Z which originates from a polyhydroxylated organic compound which is chosen from the group of etherified (C₆-C₁₈)-alkyl polysaccharides having 1 to 6 monomeric saccharide units and/or etherified aliphatic (C₆-C₁₆)-hydroxyalkylpolyols having 2 to 16 hydroxyl radicals, with the proviso that at least one of the groups R¹ or R² is a radical Z,
sulfosuccinic acid mono- and dialkyl esters having 8 to 24 carbon atoms in the alkyl group and sulfosuccinic acid monoalkyl polyoxyethyl esters having 8 to 24 carbon atoms in the alkyl group and 1 to 6 ethoxy groups,
esters of tartaric acid and citric acid with alcohols, which constitute addition products of about 2-15 molecules of ethylene oxide and/or propylene oxide onto fatty alcohols having 8 to 22 carbon atoms,
linear and branched fatty acids having 8 to 30 carbon atoms (soaps), ether carboxylic acids of the formula R—O—(CH₂—CH₂O)ₓ—CH₂—COOH, in which R is a linear alkyl group having 8 to 30 carbon atoms and x=0 or 1 to 16,
acyl sarcosinates with a linear or branched acyl radical having 6 to 22 carbon atoms and 0, 1, 2 or 3 double bonds,
acyl taurates with a linear or branched acyl radical having 6 to 22 carbon atoms and 0, 1, 2 or 3 double bonds,
acyl isethionates with a linear or branched acyl radical having 6 to 22 carbon atoms and 0, 1, 2 or 3 double bonds,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂O)_{z}—SO₃X, in which R is a preferably linear alkyl group having 8 to 30 carbon atoms, particularly preferably having 8-18 carbon atoms, z=0 or 1 to 12, particularly preferably 3, and X is a sodium, potassium, magnesium, zinc, ammonion ion or a monoalkanol, dialkanol or trialkanolammonium ion having 2 to 4 carbon atoms in the alkanol group, where a particularly preferred example is zinc cocoyl ether sulfate with a degree of ethoxylation of z=3,
mixtures of surface-active hydroxysulfonates according to DE-A-37 25 030,
sulfated hydroxyalkyl polyethylene and/or hydroxyalkylene propylene glycol ethers as in DE-A-37 23 354,
sulfonates of unsaturated fatty acids having 8 to 24 carbon atoms and 1 to 6 double bonds as in DE-A-39 26 344,
alkyl and/or alkenyl ether phosphates of the formula (V), 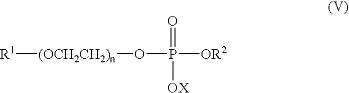
in which R¹ is preferably an aliphatic hydrocarbon radical having 8 to 30 carbon atoms, R² is hydrogen, a radical (CH₂CH₂O)ₙR¹ or X, n is numbers from 1 to 10 and X is hydrogen, an alkali metal or alkaline earth metal or NR³R⁴R⁵R⁶, where R³ to R⁶, independently of one another, are a C¹ to C₄-hydrocarbon radical,
sulfated fatty acid alkylene glycol esters of the formula R⁷CO(AlkO)ₙSO₃M, in which R⁷CO is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation, as are described in DE-A 197 36 906.5,
monoglyceride sulfates and monoglyceride ether sulfates of the formula (VI), 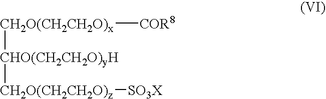
in which R⁸CO is a linear or branched acyl radical having 6 to 22 carbon atoms, x, 6 and z is in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid in the form of their sodium salts. Preference is given to using monoglyceride sulfates of the formula (VI) in which R⁸CO is a linear acyl radical having 8 to 18 carbon atoms.

In addition, the compositions according to the invention can comprise at least one protein hydrolysate or derivatives thereof. According to the invention, either vegetable or animal protein hydrolysates can be used. Animal protein hydrolysates are, for example, elastin, collagen, keratin, silk and milk protein hydrolysates, which may also be in the form of salts. According to the invention, preference is given to vegetable protein hydrolysates, e.g., soybean, wheat, almond, pea, potato and rice protein hydrolysates. Corresponding commercial products are, for example, DiaMin® (Diamalt), Gluadin® (Cognis), Lexein® (Inolex) and Crotein® (Croda).

Instead of the protein hydrolysates, it is possible to use firstly amino acid mixtures obtained by another method, secondly also individual amino acids, and physiologically compatible salts thereof. Amino acids preferred according to the invention include glycine, serine, threonine, cysteine, asparagine, glutamine, pyroglutamic acid, alanine, valine, leucine, isoleucine, proline, tryptophan, phenylalanine, methionine, aspartic acid, glutamic acid, lysine, arginine and histidine, and the zinc salts and the acid addition salts of said amino acids.

Likewise possible is the use of derivatives of the protein hydrolysates, e.g., in the form of their fatty acid condensation products. Corresponding commercial products are, for example, Lamepon® (Cognis), Gluadin® (Cognis), Lexein® (Inolex), Crolastin® or Crotein® (Croda).

According to the invention, it is also possible to use cationized protein hydrolysates, where the underlying protein hydrolysate can originate from animals, plants, marine life forms or from biotechnologically obtained protein hydrolysates. Preference is given to cationic protein hydrolysates whose underlying protein fraction has a molecular weight from 100 to 25,000 daltons, preferably 250 to 5,000 daltons. Furthermore, cationic protein hydrolysates are understood as meaning quaternized amino acids and mixtures thereof. In addition, the cationic protein hydrolysates can also be yet further derivatized. Typical examples of cationic protein hydrolysates and derivatives used according to the invention may be some of those mentioned under the INCI designations in the “International Cosmetic Ingredient Dictionary and Handbook,” (seventh edition 1997, The Cosmetic, Toiletry, and Fragrance Association 1101 17th Street, N.W., Suite 300, Washington, D.C. 20036-4702) and commercially available products: Cocodimonium Hydroxypropyl Hydrolyzed Collagen, Cocodimonium Hydroxypropyl Hydrolyzed Casein, Steardimonium Hydroxypropyl Hydrolyzed Collagen, Steardimonium Hydroxypropyl Hydrolyzed Hair Keratin, Lauryidimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin. Very particular preference is given to the cationic protein hydrolysates and derivatives based on plants.

In the compositions according to the invention, the protein hydrolysates and derivatives thereof, or the amino acids and derivatives thereof, are present in amounts to 10% by weight, based on the total composition. Amounts of from 0.1 to 5% by weight, in particular, 0.1 to 3% by weight, are particularly preferred.

In addition, the compositions according to the invention can comprise at least one mono-, oligo- or polysaccharide or derivatives thereof.

Monosaccharides suitable according to the invention are, for example, glucose, fructose, galactose, arabinose, ribose, xylose, lyxose, allose, altrose, mannose, gulose, idose and talose, the deoxy sugars fucose and rhamnose, and amino sugars such as, for example, glucosamine or galactosamine. Preference is given to glucose, fructose, galactose, arabinose and fucose; glucose is particularly preferred.

Oligosaccharides suitable according to the invention are composed of two to ten monosaccharide units, e.g., sucrose, lactose or trehalose. A particularly preferred oligosaccharide is sucrose. The use of honey, which comprises primarily glucose and sucrose, is likewise particularly preferred.

Polysaccharides suitable according to the invention are composed of more than ten monosaccharide units. Preferred polysaccharides are the starches made up of α-D-glucose units, and starch degradation products such as amylase, amylopectin and dextrins. Of particular advantage according to the invention are chemically and/or thermally modified starches, e.g., hydroxypropyl starch phosphate, dihydroxypropyl distarch phosphate or the commercial products Dry Flo®. Dextrans and their derivatives are further preferred, e.g., dextran sulfate. Likewise preferred are nonionic cellulose derivatives, such as methylcellulose, hydroxypropylcellulose or hydroxyethylcellulose, and cationic cellulose derivatives, e.g., the commercial products Celquat® and Polymer JR®, and preferably Celquat®H 100, Celquat® L 200 and Polymer JR® 400 (polyquaternium-10), and polyquaternium-24. Further preferred examples are polysaccharides of fucose units, e.g., the commercial product Fucogel®. Particular preference is given to the polysaccharides constructed from amino sugar units, in particular, chitins and their deacetylated derivatives, the chitosans, and mucopolysaccharides. The mucopolysaccharides preferred according to the invention include hyaluronic acid and its derivatives, e.g., sodium hyaluronate or dimethylsilanol hyaluronate, and chondroitin and its derivatives, e.g., chondroitin sulfate.

In one advantageous embodiment, the compositions according to the invention comprise at least one film-forming, emulsion-stabilizing, thickening or adhesive polymer chosen from natural and synthetic polymers, which may be cationic, anionic, amphoterically charged or nonionic. According to the invention, preference is given to cationic, anionic and nonionic polymers.

Among the cationic polymers, preference is given to polysiloxanes with quaternary groups, e.g., the commercial products Q2-7224 (Dow Corning), Dow Corning® 929 emulsion (with amodimethicone), SM-2059 (General Electric), SLM-55067 (Wacker), and Abil®-Quat 3270 and 3272 (Goldschmidt).

Preferred anionic polymers, which can aid the effect of the active ingredient used according to the invention, contain carboxylate and/or sulfonate groups and, as monomers, for example, acrylic acid, methacrylic acid, crotonic acid, maleic anhydride and 2-acrylamido-2-methylpropanesulfonic acid. Here, the acidic groups may be present entirely or partly as sodium, potassium, ammonium, mono- or triethanolammonium salt. Preferred monomers are 2-acrylamido-2-methylpropanesulfonic acid and acrylic acid. Very particularly preferred anionic polymers contain, as the sole monomer or as comonomer, 2-acrylamido-2-methylpropanesulfonic acid, where the sulfonic acid group may be present entirely or partly in salt form. Within this embodiment, it is preferred to use copolymers of at least one anionic monomer and at least one nonionic monomer. With regard to the anionic monomers, reference is made to the substances listed above. Preferred nonionogenic monomers are acrylamide, methacrylamide, acrylic esters, methacrylic esters, vinylpyrrolidone, vinyl ethers and vinyl esters. Preferred anionic copolymers are acrylic acid-acrylamide copolymers, and in particular, polyacrylamide copolymers with monomers containing sulfonic acid groups. A particularly preferred anionic copolymer consists of 70 to 55 mol % of acrylamide and 30 to 45 mol % of 2-acrylamido-2-methylpropanesulfonic acid, where the sulfonic acid groups are present entirely or partly as sodium, potassium, ammonium, mono- or triethanolammonium salt. This copolymer may also be present in crosslinked form, in which case the crosslinking agents used are preferably polyolefinically unsaturated compounds such as tetraallyloxyethane, allylsucrose, allylpentaerythritol and methylenebisacrylamide. One such polymer is present in the commercial product Sepigel®305 from SEPPIC. The use of this compound has proven to be particularly advantageous in the scope of the teaching according to the invention. The sodium acryloyldimethyltaurare copolymers sold under the name Simulge®600 as compound with isohexadecane and polysorbate-80 have also proven to be particularly effective according to the invention.

Further preferred anionic homopolymers and copolymers are uncrosslinked and crosslinked polyacrylic acids. Here, allyl ethers of pentaerythritol, of sucrose and of propylene may be preferred crosslinking agents. Such compounds are, for example, the commercial products Carbopol®. A particularly preferred anionic copolymer comprises, as monomer, 80-98% of an unsaturated, if desired substituted C₃₋₆-carboxylic acid or its anhydride, and 2-20% of if desired substituted acrylic esters of saturated C₁₀₋₃₀-carboxylic acids, where the copolymer may be crosslinked with the above-mentioned crosslinking agents. Corresponding commercial products are Pemulen® and the Carbopol® grades 954, 980, 1342 and ETD 2020 (ex B.F. Goodrich).

Suitable nonionic polymers are, for example, polyvinyl alcohols, which may be partially saponified, e.g., the commercial products Mowiol®, and vinylpyrrolidone/vinyl ester copolymers and polyvinylpyrrolidones, which are sold, for example, under the trade name Luviskol® (BASF).

The compositions according to the invention can also comprise at least one α-hydroxycarboxylic acid or α-ketocarboxylic acid or the ester, lactone or salt form thereof. Suitable α-hydroxycarboxylic acids or α-ketocarboxylic acids are chosen from lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2-hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, erythraric acid, threaric acid, glucaric acid, galactaric acid, mannaric acid, gularic acid, 2-hydroxy-2-methylsuccinic acid, gluconic acid, pyruvic acid, glucuronic acid and galacturonic acid. The esters of said acids are chosen from the methyl, ethyl, propyl, isopropyl, butyl, amyl, pentyl, hexyl, 2-ethylhexyl, octyl, decyl, dodecyl and hexadecyl esters. The α-hydroxycarboxylic acids or α-ketocarboxylic acids or their derivatives are present in amounts of 0.1-10% by weight, preferably 0.5-5% by weight, in each case based on the total composition.

The compositions according to the invention can comprise further active ingredients, auxiliaries and additives, for example:
vitamins, provitamins and vitamin precursors from the groups A, C, E and F, in particular, 3,4-didehydroretinol (vitamin A₂), β-carotene (provitamin of vitamin A₁), ascorbic acid (vitamin C), and the palmitic esters, glucosides or phosphates of ascorbic acid, tocopherols, in particular, α-tocopherol, and its esters, e.g., the acetate, the nicotinate, the phosphate and the succinate; also vitamin F, which is understood as meaning essential fatty acids, particularly linoleic acid, linolenic acid and arachidonic acid;
an ester of retinol (vitamin A₁) with a C₂₋₁₈-carboxylic acid, in particular, retinyl acetate or retinyl palmitate,
vitamins, provitamins or vitamin precursors of the vitamin B group or derivatives thereof, and derivatives 2-furanone, in particular, vitamin B₁ (thiamine), vitamin B₂ (riboflavin), vitamin B₃ (nicotinic acid and/or nicotinamide), vitamin B₅ (pantothenic acid and/or panthenol), vitamin B₆ (pyridoxine, pyridoxamine and/or pyridoxal) and/or vitamin B₇ (biotin),
allantoin,
antioxidants, for example, imidazoles (e.g., urocanic acid) and derivatives thereof, peptides, such as D,L-carnosine, D-carnosine, L-carnosine and derivatives thereof (e.g., anserine), chlorogenic acid and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g., buthionine sulfoximines, homocysteine sulfoximine, buthionine sulfones, penta-, hexa-, heptathionine sulfoximine) in very low tolerated doses (e.g., pmol to μmol/kg), also (metal) chelating agents (e.g., α-hydroxy fatty acids, palmitic acid, phytic acid, lactoferrin), humic acid, bile acid, bile extracts, bilirubin, biliverdin, EDTA, EGTA and derivatives thereof, unsaturated fatty acids and derivatives thereof (e.g., γ-linolenic acid, linoleic acid, oleic acid), folic acid and derivatives thereof, ubiquinone and ubiquinol and derivatives thereof, the coniferyl benzoate of benzoin resin, rutinic acid and derivatives thereof, α-glycosylrutin, ferulic acid, furfurylidene glucitol, carnosine, butylhydroxytoluene, butylhydroxyanisole, nordihydroguaiacic acid, nordihydroguaiaretic acid, trihydroxybutyrophenone, uric acid and derivatives thereof, catalase, superoxide dismutase, zinc and derivatives thereof (e.g., ZnO, ZnSO₄), selenium and derivatives thereof (e.g., selenomethionine), stilbenes and derivatives thereof (e.g., stilbene oxide, trans-stilbene oxide) and the derivatives (salts, esters, ethers, sugars, nucleotides, nucleosides, peptides and lipids) of these active ingredients suitable as antioxidant,
ceramides and pseudoceramides,
triterpenes, in particular, triterpenoic acids such as ursolic acid, rosmaric acid, betulinic acid, boswellic acid and bryonolic acid,
monomeric catechins, particularly catechin and epicatechin, leuko-anthocyanidins, catechin polymers (catechin tannins), and gallo tannins,
thickeners, e.g., gelatins, plant gums such as agar agar, guar gum, alginates, xanthan gum, gum arabic, karaya gum or carob seed flour, natural and synthetic clays and sheet silicates, e.g., bentonite, hectorite, montmorillonite or Laponite®, completely synthetic hydrocolloids, such as, for example, polyvinyl alcohol, and also Ca, Mg or Zn soaps of fatty acids,
plant glycosides,
structurants such as maleic acid and lactic acid,
dimethyl isosorbide,
alpha-, beta- and gamma-cyclodextrins, in particular, for the stabilization of retinol,
solvents, swelling and penetration substances, such as ethanol, isopropanol, ethylene glycol, propylene glycol, propylene glycol monoethyl ether, glycerol and diethylene glycol, carbonates, hydrogencarbonates, guanidines, ureas, and primary, secondary and tertiary phosphates,
perfume oils, pigments and dyes for coloring the composition,
substances for adjusting the pH, e.g., α- and β-hydroxycarboxylic acids,
complexing agents, such as EDTA, NTA, β-alaninediacetic acid and phosphonic acids,
opacifiers, such as latex, styrene/PVP and styrene/acrylamide copolymers,
pearlizing agents such as ethylene glycol monostearate and distearate and PEG-3 distearate,
propellants, such as propane/butane mixtures, N₂O, dimethyl ether, CO₂ and air,
MMP-1-inhibiting substances, in particular, chosen from photolyase and/or T4 endonuclease V, propyl gallate, precocenes, 6-hydroxy-7-methoxy-2,2-dimethyl-1 (2H)-benzopyran and 3,4-dihydro-6-hydroxy-7-methoxy-2,2-dimethyl-1 (2H)-benzopyran,
organic, mineral and/or modified mineral photoprotective filters, in particular, UVA filters and/or UVB filters.

In a preferred embodiment, the compositions according to the invention comprise at least one antiperspirant active ingredient. Suitable antiperspirant active ingredients according to the invention are water-soluble astringent or protein-coagulating metallic salts, in particular, inorganic and organic salts of aluminum, zirconium, zinc and titanium, and any mixtures of these salts. According to the invention, solubility in water is understood as meaning a solubility of at least 4 g of active substance per 100 g of solution at 20° C. According to the invention, it is possible to use, for example, alum (KAI(SO₄)₂.12H₂O), aluminum sulfate, aluminum lactate, sodium aluminum chlorohydroxylactate, aluminum chlorohydroxyallantoinate, aluminum chlorohydrate, aluminum sulfocarbolate, aluminum zirconium chlorohydrate, zinc chloride, zinc sulfocarbolate, zinc sulfate, zirconium chlorohydrate, aluminum zirconium chlorohydrate glycine complexes and complexes of basic aluminum chlorides with propylene glycol or polyethylene glycol. The liquid active ingredient preparations preferably comprise an astringent aluminum salt, in particular, aluminum chlorohydrate, and/or an aluminum zirconium compound. Aluminum chlorohydrates are sold, for example, in powder form as Micro Dry® Ultrafine or in activated form as Reach® 501 or Reach® 103 by Reheis, and also in the form of aqueous solutions as Locron® L by Clariant or as Chlorhydrol® by Reheis. An aluminum sesquichlorohydrate is supplied by Reheis under the name Reach® 301. The use of aluminum zirconium tri- or tetrachlorohydrex glycine complexes, which are available, for example, from Reheis under the name Rezal® G, is also particularly advantageous according to the invention.

The antiperspirant active ingredient is present in the compositions according to the invention in an amount of 0.01-40% by weight, preferably 2-30% by weight and in particular, 5-25% by weight, based on the amount of active substance in the total composition.

In a further particularly preferred embodiment, the compositions according to the invention comprise at least one further deodorant active ingredient besides the substance with a probiotic action. Further suitable deodorant active ingredients according to the invention are fragrances, antimicrobial, antibacterial or germicidal substances, enzyme-inhibiting substances, antioxidants and odor adsorbers.

In particular, organohalogen compounds and organohalides, quaternary ammonium compounds and zinc compounds are suitable. Preference is given to chlorhexidine and chlorhexidine gluconate, benzalkonium halides and cetylpyridinium chloride. It is also possible to use sodium bicarbonate, sodium phenolsulfonate and zinc phenolsulfonate, the constituents of linden blossom oil and of chamomile oil, bisabolol, phenoxyethanol, triclosan (Irgasan® DP300) or triethyl citrate.

As enzyme-inhibiting substances, preference is given to inhibitors for enzymes of the axillary microbe flora which are involved in the formation of body odor. These are preferably inhibitors of lipases, aryl sulfatases (see WO 01/99376), β-glucoronidases (see WO 03/039505), 5-α-reductases and aminoacylases.

Further antibacterially effective deodorant active ingredients are lantibiotics, glycoglycerolipids, sphingolipids (ceramides), sterols and other active ingredients which inhibit bacteria adhesion to the skin, e.g., glycosidases, lipases, proteases, carbohydrates, di- and oligosaccharide fatty acid esters, and alkylated mono- and oligosaccharides.

Further suitable as deodorant active ingredient are water-soluble polyols chosen from water-soluble diols, triols and polyhydric alcohols, and polyethylene glycols. Among the diols, C₂-C₁₂-diols are suitable, in particular, 1,2-propylene glycol, butylene glycols, such as, for example, 1,2-butylene glycol, 1,3-butylene glycol and 1,4-butylene glycol, pentanediols, e.g., 1,2-pentanediol, and hexanediols, e.g., 1,6-hexanediol. Also preferably suitable are glycerol and technical-grade oligoglycerol mixtures with a degree of self-condensation of from 1.5 to 10, such as, for example, technical-grade diglycerol mixtures with a diglycerol content of from 40 to 50% by weight or triglycerol, also 1,2,6-hexanetriol, and polyethylene glycols (PEG) with an average molecular weight of from 100 to 1,000 daltons, for example, PEG-400, PEG-600 or PEG-1000. Further suitable polyhydric alcohols are the C₄-, C₅- and C₆-monosaccharides and the corresponding sugar alcohols, e.g., mannitol or sorbitol.

Deodorant or antiperspirant sticks may also be in gelled form, based on anhydrous wax and based on W/O emulsions and O/W emulsions. Gel sticks can be prepared on the basis of fatty acid soaps, dibenzylidene sorbitol, N-acylamino acid amides, 12-hydroxystearic acid and other gel formers. Aerosol sprays, pump sprays, roll-on applications and creams can be present as water-in-oil emulsion, oil-in-water emulsion, silicone oil-in-water emulsion, water-in-oil microemulsion, oil-in-water microemulsion, silicone oil-in-water microemulsion, anhydrous suspension, alcoholic and hydroalcoholic solution, aqueous gel and as oil. All of the specified compositions can be thickened, for example, on the basis of fatty acid soaps, dibenzylidene sorbitol, N-acylamino acid amides, 12-hydroxystearic acid, polyacrylates of the carbomer and carbopol type, polyacrylamides and polysaccharides, which may be chemically and/or physically modified. The emulsions and microemulsions can be transparent, translucent or opaque.

Liquid and gel-like administration forms of the compositions according to the invention can comprise thickeners, e.g., cellulose ethers, such as hydroxypropylcellulose, hydroxyethylcellulose and methylhydroxypropylcellulose, thickening polymers based on polyacrylates, which may be crosslinked if desired, e.g., the carbopol grades or Pemulen® products, or polyacrylates based on polyacrylamides or containing sulfonic acid groups, e.g., Sepigel® 305 or Simulgel® EG, also inorganic thickeners, e.g., bentonites and hectorites (Laponite®).

The compositions according to the invention can comprise further cosmetically and dermatologically active ingredients, such as, for example, antiinflammatory substances, solids chosen from silicas, e.g., Aerosil® grades, silica gels, silicon dioxide, clays, e.g., bentonites or kaolin, magnesium aluminum silicates, e.g., talc, boron nitride, titanium dioxide, which may be coated if desired, optionally modified starches and starch derivatives, cellulose powders and polymer powders, also plant extracts, protein hydrolysates, vitamins, perfume oils, sebostatics, anti-acne active ingredients and keratolytics.

The compositions according to the invention can, if they are present in liquid form, be applied to flexible and absorbent carriers and be supplied as deodorant or antiperspirant cloths or sponges. Suitable flexible and absorbent carriers for the purposes of the invention are, for example, carriers made of textile fibers, collagen or polymeric foams. Textile fibers which can be used are either natural fibers, such as cellulose (cotton, linen), silk, wool, regenerated cellulose (viscose, rayon), cellulose derivatives, or else synthetic fibers, such as, for example, polyester, polyacrylonitrile, polyamide or polyolefin fibers, or mixtures of such fibers woven or nonwoven. These fibers can be processed to give absorbent cotton pads, fleeces or to give wovens or knits. Flexible and absorbent polymeric foams, e.g., polyurethane foams and polyamide foams, are also suitable substrates. The substrate can have one, two, three or more than three layers, where the individual layers can consist of the same or different materials. Each substrate layer can have a homogeneous or an inhomogeneous structure with, for example, different zones of varying density.

For the purposes of the invention, absorbent carrier substrates are regarded as being those which, at 20° C., can bind at least 10% by weight, based on the dry weight, of water in an adsorptive and/or capillary manner. However, preferably suitable carriers are those which can bind at least 100% by weight of water in an adsorptive and capillary manner.

The carrier substrates are finished by treating and/or finishing the adsorbent, flexible carrier substrates, preferably made of textile fibers, collagen or polymeric foams, with the compositions according to the invention, and optionally drying them. In this connection, the treatment (finishing) of the carrier substrates can take place by any methods, e.g., by spraying on, immersing and squeezing, soaking or simply by injecting the composition according to the invention into the carrier substrates.

Also preferred according to the invention is the administration form as aerosol, where the cosmetic composition comprises a propellant, preferably chosen from propane, butane, isobutane, pentane, isopentane, dimethyl ether, fluorocarbons and chlorofluorocarbons. A compressed propellant, such as air, nitrogen or carbon dioxide, can likewise be used. Mixtures of said propellants can likewise be used.

In one preferred embodiment, the compositions according to the invention are in the form of a liquid or solid oil-in-water emulsion, water-in-oil emulsion, multiple emulsion, microemulsion, PIT emulsion or Pickering emulsion, a hydrogel, a lipogel, a single-phase or multiphase solution, a foam, a powder or a mixture with at least one polymer suitable as medicinal adhesive. The compositions can also be administered in anhydrous form, such as, for example, an oil or a balsam. Here, the carrier may be a vegetable or animal oil, a mineral oil, a synthetic oil or a mixture of such oils.

In one particular embodiment of the compositions according to the invention, the compositions are in the form of a microemulsion. For the purposes of the invention, microemulsions are also understood as meaning the so-called “PIT” emulsions as well as the thermodynamically stable microemulsions. These emulsions are systems with the 3 components water, oil and emulsifier, which are present at room temperature as an oil-in-water emulsion. Upon heating these systems, within a certain temperature range (referred to as phase inversion temperature or “PIT”), microemulsions form which convert to water-in-oil (W/O) emulsions upon further warming. Upon subsequent cooling, O/W emulsions are again formed, although, even at room temperature, they are in the form of microemulsions or in the form of very finely divided emulsions with an average particle diameter below 400 nm and in particular, of about 100-300 nm. According to the invention, preference may be given to those microemulsions or “PIT” emulsions which have an average particle diameter of about 200 nm. Details with regard to these “PIT emulsions” are given, for example, in the publication Angew. Chem. 97, 655-669 (1985).

The examples below serve to illustrate the present invention without limiting it thereto.

Working Examples

### EXAMPLE 1

Influencing the Growth of Staphylococcus epidermidis and Staphylococcus hominis with Plant Extracts

From liquid precultures of S. epidermidis and S. hominis, cultures in LB medium with an optical density OD (600 nm) of 0.05 were inoculated. In parallel to the controls (without the addition of cells or extract), in each case 2 cultures were treated with 1% plant extract and the growth was documented over 30 h by reference to the OD measurement. After 30 h, the difference in the OD of the cultures with extract additive to the corresponding controls (without extract additive, corrected by the value without cells) was determined. The extracts of white tea (Camellia sinensis), carcade (hibiscus), mallow, grapes, grape seeds, carrot/jojoba, myrrh and calendula used selectively promote the growth of S. epidermidis with simultaneous inhibition of S. hominis.

TABLE 1
Growth effects of various plant extracts on S. epidermidis and S. hominis
(promoting or inhibiting factor after 30 h compared to the control)

| | White | | | | Grape | | Carrot/ | |
|---|---|---|---|---|---|---|---|---|
| | tea | Carcade | Mallow | Grapes | seed | Myrrh | jojoba | Calendula |
| S. epidermidis | 1.18 | 1.67 | 1.56 | 1.19 | 1.38 | 1.47 | 1.10 | 1.43 |
| S. hominis | 0.77 | 0.79 | 0.83 | 0.79 | 0.64 | 0.99 | 0.63 | 0.92 |

### EXAMPLE 2

Growth Effects of Variously Produced Hibiscus Extracts (Carcade) on S. Epidermidis and S. Hominis

From liquid precultures of S. epidermidis and S. hominis, cultures in LB medium with an optical density OD (600 nm) of 0.05 were inoculated. In parallel to the controls (without the addition of cells or extract), in each case 2 cultures were treated with 1% plant extract and the growth was documented over 30 h by reference to the OD measurement. After 30 h, the difference in the OD of the cultures with extract additive to the corresponding controls (without extract additive, corrected by the value without cells) was determined. Various hibiscus extracts from Cosmetochem were used, namely “herbasol extract unpreserved carcade in 80% PG,” “carcade (hibiscus) herbasec” and “carcade (hibiscus) herbasol extract oil-soluble.”

TABLE 2
Growth effects of variously produced hibiscus extracts (carcade) on
S. epidermidis and S. hominis (promoting or inhibiting factor after
24 h compared to isopropyl myristate)

| | Water/propylene | Water/ethanol (on | Isopropyl |
|---|---|---|---|
| | glycol | maltodextrin carrier) | myristate |
| S. epidermidis | 1.36 | 2.59 | 1.38 |
| S. hominis | 1.00 | 1.00 | 1.01 |

### EXAMPLE 3

Influencing the Growth of Staphylococcus epidermidis and Staphylococcus hominis with Myristate Derivatives

From liquid precultures of S. epidermidis and S. hominis, cultures in LB medium with an optical density OD (600 nm) of 0.05 were inoculated. In parallel to the controls (without the addition of cells or myristate), in each case 2 cultures were treated with 1% myristate derivative and the growth was documented over 8 h by reference to the OD measurement. After 8 h, the difference in the OD of the cultures with myristate additive to the corresponding controls (without myristate additive, corrected by the value without cells) was determined. Isopropyl myristate and ethyl myristate have an inhibiting action on S. hominis, with simultaneous moderate promotion of S. epidermidis.

TABLE 3
Growth effects of various myristate derivatives on S. epidermidis and
S. hominis (promoting or inhibiting factor compared to the control)

| | Isopropyl myristate | Ethyl myristate |
|---|---|---|
| S. epidermidis | 1.24 | 1.44 |
| S. hominis | 0.50 | 0.22 |

### EXAMPLE 4

Influencing the Growth of Staphylococcus epidermidis and Staphylococcus hominis with Ethylhexyl Glycerol (Sensiva SC50)

From liquid precultures of S. epidermidis and S. hominis, cultures in LB medium with an optical density OD (600 nm) of 0.05 were inoculated. In parallel to the controls [without the addition of cells or ethylhexyl glycerol (Sensiva SC 50, Schülke&Mayr)], in each case 2 cultures were treated with 0.1% ethylhexyl glycerol and the growth was documented over 24 h by reference to the OD measurement. After 4 h, 8 h and 24 h, the difference in the OD of the cultures with ethylhexyl glycerol to the corresponding controls (without extract additive, corrected by the value without cells) was determined. Ethylhexyl glycerol inhibits S. hominis, but not S. epidermidis.

TABLE 4
Growth effects of ethylhexyl glycerol over the course of time on
S. epidermidis and S. hominis (promoting or inhibiting
factor compared to the control)

| | 4 h | 8 h | 24 h |
|---|---|---|---|
| S. epidermidis | 1.06 | 1.06 | 1.10 |
| S. hominis | 0.87 | 0.92 | 0.76 |

### Example 5

Effect of Deodorant Formulations which Comprise Hibiscus Extract and Antimicrobial Components on the Growth of Staphylococcus epidermidis and Staphylococcus hominis

From overnight cultures of S. epidermidis and S. hominis, mixtures with an OD_{600=0.1} (corresponds to about 10⁴ cells/ml) in 100 ml of LB medium were prepared, to which beforehand deodorant formulations without or with 1% by weight hibiscus extract in a final concentration of 10% (v/v) were added. he cultures were then incubated for a total of 48 h at 37° C. and 100 rpm. At 0 and 48 h, the number of microbes was determined by plating out.

On account of the antimicrobially effective components, nonspecific inhibition of all types of bacteria takes place, but the antibacterial effect is synergistically overlapped by the probiotic effect of the substance with a probiotic action.

Thus, it is clear from table 5 that the deodorant formulations without hibiscus extract used have an approximately equally strong inhibiting effect on S. epidermidis and S. hominis. The considerably weaker inhibition of S. epidermidis with constantly strong inhibition of S. hominis is evident on the other hand in the case of formulations with hibiscus extract.

TABLE 5
Influence of deodorant formulations with and without hibiscus extract in
comparison

| Time | S. epidermidis | | S. hominis | |
|---|---|---|---|---|
| (h) | without hibiscus | with hibiscus | without hibiscus | with hibiscus |
| 0 | 1.41E+04 | 1.41E+04 | 4.57E+04 | 4.57E+04 |
| 48 | 7.00E+02 | 3.20E+03 | 1.00E+02 | 1.00E+02 |

### Example Formulations

Anhydrous surfactant-containing antiperspirant sticks (data in parts by weight)

| | 1.1 | 1.2 | 1.3 | 1.4 | 1.5 | 1.6 | 1.7 | 1.8 | 1.9 |
|---|---|---|---|---|---|---|---|---|---|
| Eutanol ® G 16 | 10 | — | — | 15 | 10 | — | 10 | — | 10 |
| Cetiol ® OE | — | 10 | 15 | — | — | — | — | — | — |
| Ucon Fluid ® AP | 5 | 5 | 5 | 5 | 5 | 5 | 5 | 5 | 5 |
| Cutina ® HR | 6 | 6 | 6 | 6 | 6 | 6 | 2 | 5 | 6 |
| Lorol ® C 18 | 20 | 20 | 20 | — | 20 | 20 | — | — | 20 |
| Lanette ® O | — | — | — | 20 | — | — | 10 | 12 | — |
| Eumulgin ® B 3 | 3 | 3 | 3 | 3 | 3 | 3 | 3 | 3 | — |
| Cutina ® E 24 PF | — | — | — | — | 5 | — | — | — | — |
| Aluminum chlorohydrate | 20 | 20 | 20 | 20 | 20 | 20 | 20 | — | — |
| Talc | 8 | 8 | 8 | 8 | 8 | 8 | 8 | 28 | 28 |
| Probiotic plant | 0.4 | 0.6 | 0.8 | 1.0 | 1.2 | — | — | — | 0.4 |
| extract | | | | | | | | | |
| Sensiva ® SC 50 | — | — | — | — | — | 0.4 | 0.8 | — | — |
| Isopropyl | — | — | — | — | — | — | — | 1.0 | 1.0 |
| myristate | | | | | | | | | |
| Silicone oil DC ® | ad | ad | ad | ad | ad | ad | ad | ad | ad |
| 245 | 100 | 100 | 100 | 100 | 100 | 100 | 100 | 100 | 100 |

Sprayable, translucent antiperspirant microemulsions (data in % by weight)

| | 2.1 | 2.2 | 2.3 | 2.4 | 2.5 | 2.6 | 2.7 | 2.8 |
|---|---|---|---|---|---|---|---|---|
| Plantaren ® 1200 | 1.71 | 1.71 | — | 1.71 | 1.71 | — | 1.71 | 1.71 |
| Plantaren ® 2000 | 1.14 | 1.39 | 2.40 | 1.14 | 1.39 | 2.40 | 1.14 | 1.39 |
| Glycerol monooleate | 0.71 | 0.71 | — | 0.71 | 0.71 | — | 0.71 | 0.71 |
| Dioctyl ether | 4.00 | 4.00 | 0.09 | 4.00 | 4.00 | 0.09 | 4.00 | 4.00 |
| Octyl dodecanol | 1.00 | 1.00 | 0.02 | 1.00 | 1.00 | 0.02 | 1.00 | 1.00 |
| Perfume oil | 1.00 | 1.00 | 1.00 | 1.00 | 1.00 | 1.00 | 1.00 | 1.00 |
| Aluminum chlorohydrate | 8.00 | 5.00 | 5.00 | — | — | — | 8.00 | 5.00 |
| 1,2-Propylene glycol | 5.00 | 5.00 | — | 5.00 | 5.00 | — | 5.00 | 5.00 |
| Glycerol | — | — | 5.00 | — | — | 5.00 | — | — |
| Sensiva ® SC 50 | — | — | — | — | — | — | 0.5 | 0.5 |
| Probiotic plant extract | 0.2 | 0.4 | 0.6 | 0.8 | 1.0 | 1.2 | — | 0.5 |
| Water | ad | ad | ad | ad | ad | ad | ad | ad |
| | 100 | 100 | 100 | 100 | 100 | 100 | 100 | 100 |

Soap-containing sticks (data in % by weight)

| | 3.1 | 3.2 | 3.3 | 3.4 | 3.5 | 3.6 | 3.7 | 3.8 |
|---|---|---|---|---|---|---|---|---|
| Ethanol | 22.5 | 22.5 | 22.5 | 22.5 | 22.5 | 22.5 | 22.5 | 22.5 |
| Cutina ® FS 45 | 4.4 | 4.4 | 4.4 | 4.4 | 4.4 | 4.4 | 4.4 | 4.4 |
| 1,3-Butanediol | 31.7 | 31.7 | 31.7 | 31.7 | 31.7 | 31.7 | 31.7 | 31.7 |
| 1,2-Propylene glycol | 21.0 | 21.0 | 21.0 | 21.0 | 21.0 | 21.0 | 21.0 | 21.0 |
| Eutanol ® G | 2.0 | 2.0 | 2.0 | 2.0 | 2.0 | 2.0 | 2.0 | 2.0 |
| Aethoxal ® B | 3.0 | 3.0 | 3.0 | 3.0 | 3.0 | 3.0 | 3.0 | 3.0 |
| Cremophor ® RH 455 | 0.05 | 0.05 | 0.05 | 0.05 | 0.05 | 0.05 | 0.05 | 0.05 |
| NaOH 45% strength | 1.44 | 1.44 | 1.44 | 1.44 | 1.44 | 1.44 | 1.44 | 1.44 |
| Perfume oil | 2.0 | 2.0 | 2.0 | 2.0 | 2.0 | 2.0 | 2.0 | 2.0 |
| Probiotic plant extract | 0.2 | 0.4 | 0.6 | 0.8 | 1.0 | — | — | — |
| Sensiva ® SC 50 | — | — | — | — | — | 0.3 | 0.8 | — |
| Isopropyl myristate | — | — | — | — | — | — | — | 1.0 |
| Water dist. | ad | ad | ad | ad | ad | ad | ad | ad |
| | 100 | 100 | 100 | 100 | 100 | 100 | 100 | 100 |

Soap-containing deodorant sticks (data in % by weight)

| | 3.1 | 3.2 | 3.3 | 3.4 | 3.5 | 3.6 | 3.7 | 3.8 |
|---|---|---|---|---|---|---|---|---|
| Ethanol | 22.5 | 22.5 | 22.5 | 22.5 | 22.5 | 22.5 | 22.5 | 22.5 |
| Cutina ® FS 45 | 4.4 | 4.4 | 4.4 | 4.4 | 4.4 | 4.4 | 4.4 | 4.4 |
| 1,3-Butanediol | 31.7 | 31.7 | 31.7 | 31.7 | 31.7 | 31.7 | 31.7 | 31.7 |
| 1,2-Propylene glycol | 21.0 | 21.0 | 21.0 | 21.0 | 21.0 | 21.0 | 21.0 | 21.0 |
| Eutanol ® G | 2.0 | 2.0 | 2.0 | 2.0 | 2.0 | 2.0 | 2.0 | 2.0 |
| Aethoxal ® B | 3.0 | 3.0 | 3.0 | 3.0 | 3.0 | 3.0 | 3.0 | 3.0 |
| Cremophor ® RH 455 | 0.05 | 0.05 | 0.05 | 0.05 | 0.05 | 0.05 | 0.05 | 0.05 |
| NaOH 45% strength | 1.44 | 1.44 | 1.44 | 1.44 | 1.44 | 1.44 | 1.44 | 1.44 |
| Phenoxyethanol | 1.0 | 1.0 | 1.0 | 1.0 | 1.0 | 1.0 | 1.0 | 1.0 |
| Sensiva ® SC 50 | 0.3 | 0.3 | 0.3 | 0.3 | 0.3 | 0.3 | 0.3 | 0.3 |
| Perfume oil | 2.0 | 2.0 | 2.0 | 2.0 | 2.0 | 2.0 | 2.0 | 2.0 |
| Probiotic plant extract | 0.1 | 0.3 | 0.5 | 0.8 | 1.0 | 0.5 | — | — |
| Isopropyl myristate | — | — | — | — | — | 0.8 | 0.8 | 1.0 |
| Water dist. | ad | ad | ad | ad | ad | ad | ad | ad |
| | 100 | 100 | 100 | 100 | 100 | 100 | 100 | 100 |

Deodorant in pump atomizer (data in % by weight)

| | 4.1 | 4.2 | 4.3 | 4.4 | 4.5 | 4.6 | 4.7 |
|---|---|---|---|---|---|---|---|
| Ethanol 96% strength, | 55.0 | 55.0 | 55.0 | 55.0 | 55.0 | 55.0 | 55.0 |
| (DEP denatured) | | | | | | | |
| Triethyl citrate | 3.0 | 3.0 | 3.0 | 3.0 | 3.0 | 3.0 | 3.0 |
| Cremophor ® RH 455 | 0.5 | 0.5 | 0.5 | 0.5 | 0.5 | 0.5 | 0.5 |
| Probiotic plant extract | 0.2 | 0.4 | 0.6 | 0.8 | 1.0 | — | — |
| Sensiva ® SC 50 | — | — | — | — | — | 0.4 | 0.6 |
| Perfum oil | 1.0 | 1.0 | 1.0 | 1.0 | 1.0 | 1.0 | 1.0 |
| Water | ad | ad | ad | ad | ad | ad | ad |
| | 100 | 100 | 100 | 100 | 100 | 100 | 100 |

Anhydrous deodorant spray (data in % by weight)

| | 6.1 | 6.2 | 6.3 | 6.4 |
|---|---|---|---|---|
| 2-Octyldodecanol | 0.5 | 0.5 | 0.5 | 0.5 |
| Ethanol 99% strength, | 39 | 39.45 | 39 | 39 |
| (DEP denatured) | | | | |
| Probiotic plant extract | 0.5 | 1.0 | — | — |
| Sensiva ® SC 50 | — | — | 0.5 | — |
| Isopropyl myristate | — | — | — | 1.0 |
| n-Butane | 60 | 60 | 60 | 60 |

Antiperspirant roll-on (data in % by weight)

| | 5.1 | 5.2 | 5.3 | 5.4 | 5.5 |
|---|---|---|---|---|---|
| Ethanol 96% strength, (DEP | 30.0 | 30.0 | 30.0 | 30.0 | 30.0 |
| denatured) | | | | | |
| Mergital ® CS 11 | 2.0 | 2.0 | 2.0 | 2.0 | 2.0 |
| Eumulgin ® B 3 | 2.0 | 2.0 | 2.0 | 2.0 | 2.0 |
| Aluminum chlorohydrate | 20.0 | 20.0 | 20.0 | 20.0 | 20.0 |
| Hydroxyethylcellulose | 0.5 | 0.5 | 0.5 | 0.5 | 0.5 |
| Probiotic plant extract | 0.2 | 0.5 | 1.0 | — | — |
| Sensiva ® SC 50 | — | — | — | 0.5 | — |
| Isopropyl myristate | — | — | — | — | 1.0 |
| Perfume oil | 0.8 | 0.8 | 0.8 | 0.8 | 0.8 |
| Water | ad 100 | ad | ad 100 | ad | ad 100 |
| | | 100 | | 100 | |

Antiperspirant spray of the suspension type (data in % by weight)

| | 6.1 | 6.2 | 6.3 |
|---|---|---|---|
| DC-245 | 10.0 | 10.0 | 10.0 |
| Isopropyl myristate | 5.0 | 5.0 | 5.0 |
| Aluminum chlorohydrate powder | 5.0 | 5.0 | 5.0 |
| Aerosil ® R 972 | 2.0 | 2.0 | 2.0 |
| Sensiva ® SC 50 | — | — | 0.5 |
| Probiotic plant extract | 0.5 | 1.0 | — |
| n-Butane | ad 100 | ad 100 | ad 100 |

Transparent antiperspirant gel (data in % by weight)

| | | 8.1 |
|---|---|---|
| Phase 1 | DC-245 | 7.0 |
| | DC-3225 | 10.0 |
| | Probiotic plant extract | 1.0 |
| Phase 2 | Chlorhydrol ® | 50.0 |
| | 1,2-Propylene glycol | 16.0 |
| | Water | 16.9 |

With stirring, phase 2 is added to phase 1 over the course of 25 minutes with the help of a dropping cylinder. The mixture is then stirred for 30 minutes. The mass is then homogenized uniformly for 120 seconds by moving the glass on the shear head (Ultra Turrax T50 (IKA-Werke), turrax rod, stage 8 (about 8,500 rpm)).

Antiperspirant or Deodorant Cloths

For the embodiment according to the invention as antiperspirant cloth or deodorant cloth, a single-layer substrate of 100% viscose with an aerial weight of 50 g/m² is supplied with in each case 75 g of example emulsions 2.1 or 2.2 or 2.3 per square meter or with in each case 75 g of example solutions 4.1 or 4.2, cut into cloths of suitable size and packaged in sachets.

Further example formulations (data in % by weight)

Deodorant Aerosol Basis 1

| | 1 | 2 | 3 | 4 |
|---|---|---|---|---|
| Triethyl citrate | 1.00 | 5.00 | 6.00 | 2.00 |
| Ethylhexylglycerol | 0.50 | 0.10 | 1.50 | 3.00 |
| Phenoxyethanol | 0.30 | 0.50 | 0.10 | 0.20 |
| Perfume | 0.50 | 1.50 | 1.00 | 0.50 |
| Extract from white tea | 0.05 | 0.10 | 0.15 | 0.20 |
| Aroma | 0.50 | 0.01 | 0.05 | 0.10 |
| Isopropyl myristate | 0.20 | | | |
| Marigold extract | | 0.20 | | |
| Carcade extract | | | 0.30 | |
| Myrrh extract | | | 0.10 | |
| Mixed extract from carrot and | | | | 0.20 |
| jojoba | | | | |
| Mallow extract | 0.05 | | | 0.20 |
| Hydrocarbon propellant | 85.00 | 80.00 | 70.00 | 60.00 |
| Alcohol denat. | ad 100 | ad 100 | ad 100 | ad 100 |

Deodorant Aerosol Basis 2

| | 1 | 2 | 3 | 4 |
|---|---|---|---|---|
| Isopropyl myristate | 1.00 | 10.00 | 5.00 | 2.00 |
| Phenoxyethanol | 0.30 | 0.50 | 0.10 | 0.20 |
| Perfume | 0.50 | 1.50 | 1.00 | 0.50 |
| Extract from white tea | 0.05 | | 0.05 | |
| Ethylhexylglycerol | 0.50 | 0.10 | 1.50 | 3.00 |
| Grape extract | 0.05 | | | |
| Marigold extract | | 0.10 | | |
| Carcade extract | | | 0.15 | 0.50 |
| Hydrocarbon propellant | 75.00 | 85.00 | 78.00 | 60.00 |
| Alcohol denat. | ad 100 | ad 100 | ad 100 | ad 100 |

Antiperspirant Aerosol Basis 1

| | 1 | 2 | 3 | 4 |
|---|---|---|---|---|
| Aluminum chlorohydrate | 4.00 | 10.00 | | |
| Aluminum chlorohydrate activated | | | 2.00 | 10.00 |
| Disteardimonium hectorite | 0.50 | 1.50 | 0.80 | 1.20 |
| propylene carbonate | | | | |
| Perfume | 0.80 | 0.50 | 1.00 | 1.50 |
| Encapsulated perfume/active | 1.50 | 0.10 | 1.50 | 0.10 |
| (firecaps) | | | | |
| Ethylhexylglycerol | 0.50 | | | 0.25 |
| Isopropyl myristate | | 1.00 | | |
| Mixed extract from carrot and | 0.10 | | | |
| jojoba | | | | |
| Myrrh extract | | 0.20 | | |
| Extract from white tea | | | 0.30 | |
| Carcade extract | | | | 0.60 |
| Hydrocarbon propellant | 85.00 | 75.00 | 80.00 | 60.00 |
| Cyclopentasiloxane/ | ad 100 | ad 100 | ad 100 | ad 100 |
| cyclohexasiloxane | | | | |

Antiperspirant Aerosol Basis 2

| | 1 | 2 | 3 | 4 |
|---|---|---|---|---|
| Aluminum chlorohydrate | 4.00 | 10.00 | | |
| Aluminum chlorohydrate activated | | | 2.00 | 10.00 |
| Disteardimonium hectorite | 0.50 | 1.50 | 0.80 | 1.20 |
| propylene | | | | |
| carbonate | | | | |
| Perfume | 0.80 | 0.50 | 1.00 | 1.50 |
| Aroma | 0.50 | 0.01 | 0.05 | 0.10 |
| Di-C12-13-alkylmalate | | 0.50 | 0.50 | 10.00 |
| Ethylhexyl palmitate | ad 100 | 5.00 | | |
| Ethylhexylglycerol | 0.50 | | | |
| Isopropyl myristate | | 0.20 | | |
| Extract from white tea | | | 0.40 | |
| Mallow extract | | | | 0.65 |
| Hydrocarbon propellant | 85.00 | 75.00 | 80.00 | 60.00 |
| Cyclopentasiloxane/ | | ad 100 | ad 100 | ad 100 |
| cyclohexasiloxane | | | | |

Roll-on alcoholic

| | 1 | 2 | 3 | 4 | 5 | 6 |
|---|---|---|---|---|---|---|
| Denat. alcohol | 35.00 | 30.00 | 28.00 | 30.00 | 30.00 | 40.00 |
| Aluminum chlorohydrate 50% solution | 16.00 | 40.00 | 16.00 | 16.00 | 40.00 | |
| Aluminum zirconium | | | | | | 45.00 |
| pentachlorohydrate 40% solution | | | | | | |
| Ceteareth-12 | 2.50 | 1.50 | 2.00 | 2.00 | | 2.50 |
| Ceteareth-30 | 2.50 | 2.00 | 1.50 | 2.00 | | 2.50 |
| PEG-40 hydrogenated castor oil | | | | | 3.00 | |
| Perfume | 0.70 | 1.00 | 1.50 | 1.20 | 1.00 | 1.20 |
| Tocopheryl acetate | 0.05 | 0.10 | | 0.25 | | 0.05 |
| Hydroxyethylcellulose | 0.50 | 0.30 | 0.40 | 0.60 | 0.30 | 0.50 |
| Zinc gluconate | | 0.10 | | | 0.10 | 0.10 |
| Colors approved for cosmetics | 0.0005 | 0.0010 | 0.0005 | 0.0100 | 0.0001 | |
| Grape seed extract | 0.20 | 0.50 | | | | |
| Carcade extract | | | 0.30 | 0.60 | | |
| Ethylhexylglycerol | | | | | 0.30 | 0.70 |
| Aqua | ad 100 | ad 100 | ad 100 | ad 100 | ad 100 | ad 100 |

Roll-on emulsion

| | 1 | 2 | 3 | 4 | 5 | 6 |
|---|---|---|---|---|---|---|
| Steareth-2 | 2.50 | 3.00 | 3.00 | 2.80 | 2.80 | 2.80 |
| PPG-15 stearyl ether | 2.00 | 3.00 | 2.00 | 2.20 | 2.00 | 2.00 |
| Steareth-21 | 1.00 | 1.00 | 3.00 | 1.00 | 1.30 | 1.30 |
| Aluminum chlorohydrate 50% solution | 40.00 | 40.00 | 40.00 | 40.00 | 40.00 | |
| Aluminum tetrachlorohydrate glyc | | | | | | 63.00 |
| 35% solution | | | | | | |
| Allantoin | 0.10 | | 0.10 | | 0.10 | |
| Tocopheryl acetate | 0.05 | 0.05 | 0.05 | 0.25 | 0.25 | 0.25 |
| Perfume | 1.00 | 1.50 | 1.30 | 0.80 | 1.00 | 1.20 |
| Ethylhexylglycerol | 0.10 | 0.50 | | | | 0.20 |
| Isopropyl myristate | | | 1.00 | | | |
| Myrrh extract | | | | 0.20 | 0.50 | 0.80 |
| Aqua | ad 100 | ad 100 | ad 100 | ad 100 | ad 100 | ad 100 |

Deodorant stick/soap basis - alcoholic

| | 1 | 2 | 3 | 4 |
|---|---|---|---|---|
| Alcohol denat. | 40.00 | 40.00 | 35.00 | 30.00 |
| 1,2-Propylene glycol | 30.32 | 32.32 | 32.32 | 38.00 |
| 1,3-Butylene glycol | 12.00 | 10.00 | 15.00 | 12.00 |
| Sodium palmitate | 3.10 | 3.50 | 2.80 | 3.10 |
| Sodium stearate | 3.10 | 3.50 | 2.80 | 3.10 |
| Glycerol 86% | 2.00 | 1.00 | 1.70 | |
| PPG-5 laureth-5 | 0.50 | 1.00 | 1.00 | 0.50 |
| Perfume | 1.00 | 0.60 | 1.30 | 1.00 |
| Octyldodecanol | 1.00 | 0.50 | 1.00 | 0.70 |
| Phenoxyethanol | 1.00 | 0.50 | 1.00 | 0.50 |
| Ethylhexylglycerol | 0.50 | | 0.30 | |
| Tocopheryl acetate | 0.05 | | 0.10 | 0.25 |
| PEG-40 hydrogenated castor oil | 0.02 | 1.00 | | 0.10 |
| isopropyl myristate | | | | |
| Carcade extract | 0.20 | | 0.30 | |
| Mallow extract | | | 0.20 | |
| Mixed extract from carrot | | | | 0.80 |
| and jojoba | | | | |
| Aqua | ad 100 | ad 100 | ad 100 | ad 100 |

Deodorant stick/soap basis - nonalcoholic

| | 1 | 2 | 3 | 4 |
|---|---|---|---|---|
| PEG-8 | 40.00 | 45.00 | 50.00 | 46.00 |
| Sodium palmitate | 2.50 | 2.50 | 2.50 | 2.50 |
| Sodium stearate | 2.50 | 2.50 | 2.50 | 2.50 |
| 1,3-Butylene glycol | 5.00 | 2.00 | 3.00 | 4.00 |
| PEG-14 dimethicone | 1.00 | 2.00 | 1.50 | 1.50 |
| Phenoxyethanol | 1.00 | 2.00 | 0.50 | 1.00 |
| Perfume | 1.00 | 1.20 | 0.80 | 1.00 |
| Ethylhexylglycerol | 0.30 | | 0.30 | |
| Steareth-10 | 0.20 | | 0.20 | 0.20 |
| Mixed extract from carrot | 0.60 | | | |
| and jojoba | | | | |
| Mallow extract | | 0.75 | | |
| Carcade extract | | | 0.30 | |
| Isopropyl myristate | | | | 1.0 |
| Aqua | ad 100 | ad 100 | ad 100 | ad 100 |

Antiperspirant stick base 1

| | 1 | 2 | 3 | 4 |
|---|---|---|---|---|
| PPG-14 butyl ether | 15.00 | 18.00 | 12.00 | 19.00 |
| Hydrogenated castor oil | 1.00 | 1.50 | 2.00 | 1.50 |
| Stearyl alcohol | 20.00 | 18.00 | 15.00 | 18.00 |
| Ceteareth-30 | 3.00 | 2.00 | 4.00 | |
| Isoceteth-20 | | | | 2.50 |
| Perfume | 1.00 | 1.20 | 0.80 | 1.50 |
| Aluminum chlorohydrate | 20.00 | 22.00 | 18.00 | |
| Aluminum zirconium | | | | 22.00 |
| tetrachlorohydrate glyc | | | | |
| Allantoin | 0.10 | | | 0.10 |
| Cocoglycerides | 4.00 | 6.00 | 3.00 | 5.00 |
| Talc | 3.00 | 2.00 | 5.00 | 3.00 |
| Tocopherol acetate | 0.20 | | 0.50 | 0.10 |
| Ethylhexylglycerol | 0.50 | | | |
| Isopropyl myristate | | 1.00 | | |
| Carcade extract | | | 0.35 | |
| Mallow extract | | | | 0.50 |
| Cyclopentanesiloxane | ad 100 | ad 100 | ad 100 | ad 100 |

Antiperspirant stick base 2

| | 1 | 2 | 3 | 4 |
|---|---|---|---|---|
| Hexyldecanol | 10.00 | 12.00 | 10.00 | 8.00 |
| PPG-14 butyl ether | 6.00 | 5.00 | 6.00 | 8.00 |
| Hydrogenated castor oil | 4.00 | 5.00 | 6.00 | 5.00 |
| Stearyl alcohol | 12.00 | 14.00 | 11.00 | 16.00 |
| Cetyl alcohol | 6.00 | 5.00 | 6.00 | 3.00 |
| PEG-20 glyceryl stearate | 5.00 | 4.00 | 6.00 | 4.00 |
| Ceteareth-30 | 3.00 | 1.00 | 3.00 | |
| Perfume | 1.00 | 1.20 | 0.80 | 1.00 |
| Aluminum chlorohydrate | 20.00 | 20.00 | 18.00 | |
| Aluminum zirconium | | | | 23.00 |
| tetrachlorohydrate glyc | | | | |
| Talc | 8.00 | 5.00 | 8.00 | 7.00 |
| Ethylhexylglycerol | 0.20 | 0.80 | | |
| Tocopherol acetate | 0.25 | | 0.50 | |
| Extract from white tea | 0.30 | | | |
| Isopropyl myristate | | | 1.00 | |
| Carcade extract | | | 0.10 | 1.00 |
| Cyclopentasiloxane | ad 100 | ad 100 | ad 100 | ad 100 |

Deodorant pump dispenser

| | 1 | 2 | 3 | 4 |
|---|---|---|---|---|
| Alcohol denat. | 50.00 | 55.00 | 60.00 | 40.00 |
| Triethyl citrate | 2.50 | 3.50 | 4.00 | 3.00 |
| PEG-40 hydrogenated castor oil | 1.00 | 0.50 | 0.50 | 2.00 |
| Ethylhexylglycerol | 0.10 | 0.30 | | |
| Tocopheryl acetate | 0.05 | 0.20 | 0.10 | |
| Benzophenone-2 | 0.01 | 0.01 | 0.01 | 0.05 |
| Colors approved for cosmetics | 0.0001 | 0.0005 | 0.0010 | |
| Perfume | 0.80 | 1.00 | 2.00 | 1.50 |
| Grape seed extract | 0.20 | | | |
| Myrrh extract | | 0.15 | | |
| Carcade extract | | 0.15 | | |
| Marigold extract | | | 0.35 | |
| Extract from white tea | | | | 0.45 |
| Aqua | ad 100 | ad 100 | ad 100 | ad 100 |

Antiperspirant pump dispenser (PIT technology)

| | 1 | 2 | 3 | 4 |
|---|---|---|---|---|
| Aluminum chlorohydrate 50% | 30.00 | 40.00 | 35.00 | 40.00 |
| solution | | | | |
| Dicaprylyl ether | 10.00 | 10.00 | 8.00 | 9.00 |
| Glycerol 86% | 5.00 | 3.00 | 5.00 | 3.00 |
| Beheneth-10 | 3.30 | 4.00 | 3.50 | 4.00 |
| Cetearyl isononanoate | | | 4.00 | 5.00 |
| Hexyldecanol/hexyldecyl laurate | 3.00 | 5.00 | | |
| Perfume | 1.00 | 0.80 | 1.20 | 1.00 |
| Polysorbate 20/linoleic acid | 0.20 | 0.20 | 0.50 | |
| Allantoin | 0.10 | | | 0.20 |
| Ethylhexylglycerol | 0.50 | | | |
| Isopropyl myristate | | 0.20 | | |
| Grape seed extract | | | 0.50 | |
| Mixed extract from carrot and | | | | 0.50 |
| jojoba | | | | |
| Aqua | ad 100 | ad 100 | ad 100 | ad 100 |

Deodorant cloth

| | 1 | 2 | 3 | 4 |
|---|---|---|---|---|
| Alcohol denat. | 50.00 | 55.00 | 60.00 | 40.00 |
| Triethyl citrate | 2.50 | 3.50 | 4.00 | 3.00 |
| PEG-40 hydrogenated castor oil | 1.00 | 0.50 | 0.50 | 2.00 |
| Ethylhexylglycerol | 0.10 | 0.30 | | |
| Tocopheryl acetate | 0.05 | 0.20 | 0.10 | |
| Benzophenone-2 | 0.01 | 0.01 | 0.01 | 0.05 |
| Colors approved for cosmetics | 0.0001 | 0.0005 | 0.0010 | |
| Perfume | 0.80 | 1.00 | 2.00 | 1.50 |
| Grape seed extract | 0.25 | | | |
| Mallow extract | | 0.30 | | |
| Talc | 3.00 | 2.00 | 5.00 | 3.00 |
| Carcade extract | 0.15 | | | |
| Grape extract | | | 0.50 | |
| Marigold extract | | | | 0.40 |
| Aqua | ad 100 | ad 100 | ad 100 | ad 100 |

Antiperspirant cloth (PIT technology)

| | 1 | 2 | 3 | 4 |
|---|---|---|---|---|
| Aluminum chlorohydrate 50% | 30.00 | 40.00 | 35.00 | 40.00 |
| solution | | | | |
| Dicaprylyl ether | 10.00 | 10.00 | 8.00 | 9.00 |
| Glycerol 86% | 5.00 | 3.00 | 5.00 | 3.00 |
| Beheneth-10 | 3.30 | 4.00 | 3.50 | 4.00 |
| Cetearyl isononanoate | | | 4.00 | 5.00 |
| Hexyldecanol/hexyldecyl laurate | 3.00 | 5.00 | | |
| Perfume | 1.00 | 0.80 | 1.20 | 1.00 |
| Polysorbate 20/linoleic acid | 0.20 | 0.20 | 0.50 | |
| Allantoin | 0.10 | | | 0.20 |
| Grape extract | 0.25 | 0.40 | | |
| Carcade extract | 0.25 | | 0.50 | |
| Mallow extract | | | | 0.65 |
| Preservative system | 0.50 | 0.20 | 1.00 | 0.50 |
| Aqua | ad 100 | ad 100 | ad 100 | ad 100 |

Clear antiperspirant gel

| | 1 | 2 | 3 | 4 |
|---|---|---|---|---|
| 1,2-Propylene glycol | 18.00 | 23.00 | 18.00 | 20.00 |
| Aluminum chlorohydrate 50% | 40.00 | 40.00 | 40.00 | 40.00 |
| solution | | | | |
| Cyclopentasiloxane | 14.20 | 14.20 | 14.20 | 14.20 |
| Alcohol denat. | 5.00 | 10.00 | 8.00 | 10.00 |
| BIS-PEG/PPG-14/14 dimethicone | 3.50 | 2.50 | 3.20 | 3.00 |
| Perfume | 0.60 | 0.60 | 1.00 | 1.30 |
| Allantoin | | 0.10 | | |
| Ethylhexylglycerol | 0.50 | | | |
| Marigold extract | 0.20 | | | |
| Grape seed extract | | 0.35 | | |
| Carcade extract | | | 0.50 | |
| Myrrh extract | | | | 0.85 |
| Aqua | ad 100 | ad 100 | ad 100 | ad 100 |

Clear deodorant gel

| | 1 | 2 | 3 | 4 |
|---|---|---|---|---|
| Alcohol denat. | 30.00 | 40.00 | 50.00 | 60.00 |
| Ceteareth-12 | 1.50 | | 2.00 | |
| Ceteareth-20 | 2.50 | | 2.00 | |
| PEG-40 hydrogenated castor oil | | 3.00 | | 2.00 |
| Carbomer | 0.30 | 0.50 | 0.80 | 1.00 |
| Perfume | 0.60 | 0.60 | 1.00 | 1.30 |
| Mallow extract | 0.10 | | | |
| Myrrh extract | | 0.20 | | |
| Grape seed extract | 0.25 | | | 0.60 |
| Mixed extract of carrot and jojoba | | 0.15 | | |
| Carcade extract | | | 0.35 | |
| Aqua | ad 100 | ad 100 | ad 100 | ad 100 |

In order to achieve clear gels, the refractive index of the water phase is matched to the refractive index of the oil phase, water or propylene glycol serving as variable. The thickener (carbomer) is to be adjusted to the desired pH with a suitable neutralizing agent (TEA, AMP, NaOH, LiOH).

Antiperspirant cream

| | 1 | 2 | 3 | 4 |
|---|---|---|---|---|
| Aluminum chlorohydrate 50% | 40.00 | 40.00 | 35.00 | 45.00 |
| solution | | | | |
| Glyceryl stearate | 5.00 | 4.50 | 5.50 | 6.00 |
| Cetyl alcohol | 2.00 | 1.50 | 3.00 | 1.50 |
| Behenyl alcohol | 1.50 | 4.00 | 3.50 | 5.00 |
| Dimethicone | 2.00 | 1.50 | 2.50 | 3.00 |
| Ceteareth-12 | 1.50 | 2.00 | 2.50 | 1.30 |
| Ceteareth-20 | 1.50 | 2.00 | 2.50 | 1.30 |
| Hexyldecanol/hexyldecyl laurate | 3.00 | 4.00 | 2.50 | 2.40 |
| Cyclopentasiloxane | 1.50 | 3.00 | 2.00 | 1.00 |
| Tocopheryl acetate | 0.05 | 0.25 | | |
| Perfume | 0.80 | 1.00 | 1.50 | 2.00 |
| Allantoin | 0.10 | 0.10 | | |
| Preservative system | 0.05 | 0.05 | 0.05 | 0.05 |
| Ethylhexylglycerol | 0.5 | | | |
| Isopropyl myristate | | 1.0 | | |
| Carcade extract | | 0.2 | 0.5 | 0.8 |
| Aqua | ad 100 | ad 100 | ad 100 | ad 100 |

Antiperspirant cream anhydrous soft solid

| | 1 | 2 | 3 | 4 |
|---|---|---|---|---|
| Aluminum chlorohydrate | 20.00 | 22.00 | 20.00 | |
| Aluminum zirconium | | | | 24.00 |
| tetrachlorohydrate glyc | | | | |
| Hexyldecanol | 5.00 | 4.50 | 5.50 | 6.00 |
| Dicaprylyl ether | 3.00 | 4.00 | 3.50 | 5.00 |
| Cocoglyceride | 5.00 | 6.00 | 7.00 | 3.00 |
| C18-C36 triglycerides | 6.00 | 5.00 | 4.00 | 3.00 |
| Ceteareth-30 | 3.00 | 2.00 | 2.50 | 4.00 |
| PEG-20 glyceryl stearate | 5.00 | 6.00 | 3.00 | 2.00 |
| Cellulose | 3.00 | 2.00 | 5.00 | 1.00 |
| Aluminum starch octenylsuccinate | 5.00 | 4.00 | 6.00 | 5.00 |
| Silica | 1.00 | 2.00 | 0.50 | |
| Talc | 10.00 | 5.00 | 7.00 | 12.00 |
| Allantoin | 0.10 | 0.10 | | |
| Perfume | 1.00 | 1.50 | 2.00 | 0.80 |
| Ginseng extract | 0.05 | 0.20 | 0.50 | |
| Marigold extract | 0.1 | | | |
| Myrrh extract | | 0.3 | | |
| Grape extract | | | 0.5 | 1.0 |
| Cyclopentasiloxane | ad 100 | ad 100 | ad 100 | ad 100 |

Deodorant/antiperspirant powder

| | 1 | 2 | 3 | 4 |
|---|---|---|---|---|
| Aluminum chlorohydrate | 20.00 | | | |
| Aluminum zirconium | | | | 24.00 |
| tetrachlorohydrate glyc | | | | |
| Silica | 2.00 | 2.00 | 1.00 | 1.00 |
| Triclosan | | 0.30 | 0.10 | |
| Sensiva SC 50 | | | 1.00 | |
| Perfume | 1.00 | 0.50 | 2.00 | 1.00 |
| Extract from green tea | 0.05 | | 0.50 | |
| Extract from white tea | 0.1 | | | |
| Grape extract | | 0.2 | 0.5 | |
| Carcade extract | | | 0.1 | 0.6 |
| Talc | ad 100 | ad 100 | ad 100 | ad 100 |

Deodorant soap

| | 1 | 2 | 3 | 4 |
|---|---|---|---|---|
| Sodium tallowate | 55.00 | | 60.00 | |
| Sodium palmitate | | 55.00 | | 60.00 |
| Sodium cocoate | 22.00 | | 27.00 | |
| Sodium palm oleate | | 22.00 | | 27.00 |
| Talc | 10.00 | 10.00 | | |
| Lauryl glucoside | | 2.00 | | 2.00 |
| Perfume | 1.00 | 1.00 | 1.50 | 0.50 |
| Sodium chloride | 0.50 | 0.50 | 0.50 | 0.50 |
| Tetrasodium EDTA | 0.30 | 0.20 | 0.30 | 0.10 |
| Tocopherol | 0.10 | 0.30 | | |
| Antibacterial active | 0.30 | | 0.50 | |
| Colors approved for cosmetics | 0.01 | 0.05 | 0.03 | |
| Ethylhexylglycerol | 0.2 | 0.5 | | |
| Isopropyl myristate | | | 1.0 | |
| Carcade extract | | | | 0.6 |
| Aqua | ad 100 | ad 100 | ad 100 | ad 100 |

Deodorant syndet

| | 1 | 2 | 3 | 4 |
|---|---|---|---|---|
| Sodium laureth sulfate | 30.00 | 30.00 | 25.00 | 30.00 |
| Sodium cocoyl isethionate | 15.00 | 12.00 | 20.00 | 15.00 |
| Disodium laureth sulfosuccinate | 10.00 | 15.00 | 10.00 | 8.00 |
| Cetylstearyl alcohol | 10.00 | 12.00 | 10.00 | 10.00 |
| Stearic acid | 10.00 | 10.00 | 12.00 | 8.00 |
| Starch | 10.00 | 2.00 | 10.00 | 5.00 |
| Talc | 2.00 | 10.00 | | 5.00 |
| Perfume | 0.50 | 1.00 | 0.30 | 0.80 |
| Tetrasodium EDTA | 0.30 | 0.20 | 0.30 | 0.10 |
| Tocopherol acetate | 0.10 | 0.30 | | |
| Antibacterial | 0.30 | | 0.50 | |
| Colors approved for cosmetics | 0.01 | 0.05 | 0.03 | |
| Mixed extract of carrot and jojoba | 0.2 | 0.5 | | |
| Extract from mallow flowers | | | 0.5 | |
| Extract from leaves of white tea | | | | 1.0 |
| Aqua | ad 100 | ad 100 | ad 100 | ad 100 |

Deodorant washing lotion

| | 1 | 2 | 3 | 4 |
|---|---|---|---|---|
| Sodium laureth sulfate | | 5.00 | 4.00 | |
| Disodium laureth sulfosuccinate | 6.00 | | 2.00 | 8.00 |
| Lauryl glucoside | 4.00 | 5.00 | 4.00 | 4.00 |
| Potassium cocoyl hydrolyzed | 2.00 | 3.00 | 5.00 | 2.00 |
| collagen | | | | |
| PEG-7 glyceryl cocoate | 3.00 | 3.00 | 5.00 | 3.00 |
| PEG-120 methyl glucose dioleate | 1.00 | 2.00 | 3.00 | 1.00 |
| Perfume | 0.30 | 0.50 | 0.80 | 0.50 |
| Tetrasodium EDTA | 0.30 | 0.20 | | 0.10 |
| Tocopherol acetate | 0.10 | 0.30 | | |
| Antibacterial | 0.30 | | 0.50 | |
| Citric acid | 0.20 | 0.30 | 0.20 | 0.30 |
| Colors approved for cosmetics | 0.01 | 0.05 | 0.03 | |
| Ethylhexylglycerol | 0.2 | 0.5 | | |
| Grape seed extract | | | 0.5 | |
| Carcade extract | | | | 0.8 |
| Aqua | ad 100 | ad 100 | ad 100 | ad 100 |

List of raw materials used

| Raw material | Supplier | INCI nomenclature |
|---|---|---|
| Allantoin | Merck | Allantoin |
| Rezal G solution | Reheis | Aluminum Tetrachlorohydrate Glyc 35% |
| | | solution |
| Rezal 36 GP SUF | Reheis | Aluminum zirconium tetrachlorohydrate Glyc |
| Locron L | Clariant | Aluminum chlorohydrate 50% solution |
| Microdry | Reheis | Aluminum chlorohydrate |
| Microdry UF | Reheis | Aluminum chlorohydrate |
| Chlorhydrol solution | Reheis | Aluminum chlorohydrate 50% solution |
| Reach 103 | Reheis | Aluminum chlorohydrate activated |
| Rezal 67 | Reheis | Aluminum Zirconium Pentachlorohydrate |
| | | 40% solution |
| Dry Flo PC | National Starch | Aluminum-Starch-Octenylsuccinate |
| Cooling agent | Different | Aroma |
| Mergital B10 | Cognis | Beheneth-10 |
| Stenol 1822 A | Cognis | Behenyl Alcohol |
| Uvinul D 50 | BASF | Benzophenone-2 |
| Abil EM 97 | Degussa | BIS-PEG/PPG-14/14 Dimethicone |
| Synchrowax HGLC | Croda | C18-C36 Triglyceride |
| Carbopol ETD 2001 | Noveon | Carbomer |
| Vitacel L-600-20 FCC | Rettenmaier | Cellulose |
| Mergital CS 11 | Cognis | Ceteareth-11 |
| Eumulgin B1 | Cognis | Ceteareth-12 |
| Eumulgin B2 | Cognis | Ceteareth-20 |
| Eumulgin B3 | Cognis | Ceteareth-30 |
| Lanette O | Cognis | Cetearyl Alcohol |
| Cetiol SN | Cognis | Cetearyl Isononanoate |
| Lorol C16 | Cognis | Cetyl Alcohol |
| Novata AB | Cognis | Cocoglycerides |
| dc 245 | Dow Corning | Cyclopentasiloxane |
| Dc 3225 | Dow Corning | Cyclomethicone/Dimethicone Copolyol |
| dc 345 | Dow Corning | Cyclopentasiloxane/Cyclohexasiloxane |
| Cosmacol EMI | Condea | di-C12-13 Alkyl Malate |
| Cetiol OE | Cognis | Dicapryl Ether |
| Baysilone M350 | Bayer | Dimethicone |
| Powder quality | Different | Disodium Laureth Sulfosuccinate |
| Texapon SB 3 UP | Cognis | Disodium Laureth Sulfosuccinate |
| Bentone Gel VS 5 PCV | Rheox | Disteardimonium Hectorite Propylene |
| | | Carbonate |
| Fircaps | Firmenich | Encapsulated Perfume/Active (Fircaps) |
| Sensiva SC 50 | Schülke & Mayr | Ethylhexylglycerol |
| Cegesoft C24 | Cognis | Ethylhexyl palmitate |
| Cutina MD-V | Cognis | Glyceryl Stearate |
| Eutanol G 16 | Cognis | Hexyldecanol |
| Cetiol PGL | Cognis | Hexyldecanol/hexyldecyl Laurate |
| Drivosol | Hüls | Hydrocarbon Propellant |
| Cutina HR | Cognis | Hydrogenated Castor Oil |
| Natrosol 250 HR | Hercules Aqualon | Hydroxyethylcellulose |
| Arlasolv 200 | Uniqema | Isoceteth-20 |
| Isopropyl myristate | Cognis | Isopropyl Myristate |
| Plantacare 1200 UP | Cognis | Lauryl Glucoside |
| Eutanol G | Cognis | Octyldodecanol |
| Perfume | Different | Perfume |
| Glucamte DOE 120 | Amerchol | PEG-120 Methyl Glucose Dioleate |
| Abil B 8843 | Degussa | PEG-14 Dimethicone |
| Cutina E 24 | Cognis | PEG-20 Glyceryl Stearate |
| Eumulgin HRE 40 | Cognis | PEG-40 Hydrogenated Castor Oil |
| Cetiol HE | Cognis | PEG-7 Glyceryl Cocoate |
| Phenoxyethanol | Bayer | Phenoxyethanol |
| Plant extract | Different | Plant Extract |
| Vitamin F water soluble | Crodarom | Polysorbate 20/Linoleic Acid |
| Lamepon S | Cognis | Potassium Cocoyl Hydrolyzed Collagen |
| Ucon Fluid AP | Ucon | PPG-14 Butyl Ether |
| Arlamol E | Uniqema | PPG-15 Stearyl Ether |
| Aethoxal B | Cognis | PPG-5 Laureth-5 |
| Aerosil 200 | Degussa | Silica |
| Aerosil 200 | Degussa | Silica |
| Aerosol R 972 | Degussa | Silica Dimethyl Silylate |
| Soap based on Edenor | Cognis | Sodium Cocoate |
| K12-18 | | |
| Powder quality | Different | Sodium Laureth Sulfate |
| Texapon NSO UP | Cognis | Sodium Laureth Sulfate |
| Soap based on Edenor | Cognis | Sodium Palm Oleate |
| Soap based on Cutina | Cognis | Sodium Palmitate |
| FS 45 | | |
| Soap based on Edenor | Cognis | Sodium Palmitate |
| C16-98/100 | | |
| Soap based on Cutina | Cognis | Sodium Stearate |
| FS 45 | | |
| Soap based on Edenor TIS | Cognis | Sodium Tallowate |
| GAH | | |
| Powder quality | Different | Sodium Cocoyl Isethionate |
| Brij 76 | Uniqema | Steareth-10 |
| Brij 72 | Uniqema | Steareth-12 |
| Brij 721 | Uniqema | Steareth-21 |
| Lorol C18 | Cognis | Stearyl Alcohol |
| Lorol C18 | Cognis | Stearyl Alcohol |
| Steasilk 5 GGHT | Luzenac | Talc |
| Talcum Pharma G | Grolmann | Talc |
| Trilon B liq. | BASF | Tetrasodium EDTA |
| Vitamin E | Roche | Tocopherol |
| Vitamin E acetate | BASF | Tocopheryl Acetate |
| Irgasan DP 300 | Ciba Spec. | Triclosan |
| Citrofol AL | Jungbunzlauer | Triethyl Citrate |
| Zinc gluconate | Interorgana | Zinc Gluconate |
| Leaf extract from white tea | | Cosmetochem |
| Carcade extract | | Cosmetochem |
| Flower extract from mallow | | Cosmetochem |
| Grape extract | | Cosmetochem |
| Mixed extract from carrot and jojoba | | Cosmetochem |
| Myrrh extract | | Cosmetochem |
| Marigold extract | | Cosmetochem |
| Grape seed extract | | Cosmetochem |
| Green tea extract | | Dragoco |

1. A method for promoting probiotic activity on the skin comprising contacting the skin with a probiotic effective amount of a substance having a probiotic effect selected from the group consisting of a plant extract, a glycerol monoalkyl ether, and a fatty acid ester or a combination of the plant extract and the glycerol monoalkyl ether, whereby the growth of desired skin microbes is promoted and the growth of undesired skin microbes is inhibited.

2. The method of claim 1, wherein the desired skin microbes are saprophytic skin microbes.

3. The method of claim 1, wherein the desired skin microbes are odor-neutral skin microbes.

4. The method of claim 3, wherein the odor-neutral skin microbes are odor-neutral Staphylococci.

5. The method of claim 4, wherein the odor-neutral Staphylococci are S. epidermidis.

6. The method of claim 1, wherein the undesired microbes are odor-forming microbes.

7. The method of claim 6, wherein the odor-forming microbes are odor-forming forming Staphylococci, Gram-positive anaerobic cocci, odor-forming coryne bacteria or odor-forming micrococci.

8. The method of claim 7, wherein the odor-forming Staphylococci are Staphylococcus hominis.

9. The method of claim 7, wherein the Gram-positive anaerobic cocci are Anaerococcus octavius.

10. The method of claim 1, wherein the skin is the skin in the axillary area.

11. The method of claim 1, wherein the substance with a probiotic action is a plant extract.

12. The method of claim 11, wherein the plant extract is a tea extract, an extract from the group of Vitaceae, Asteraceae, Apiaceae or Buxaceae or mixtures thereof.

13. The method of claim 12, wherein the tea extract is Theaceae or Malvaceae.

14. The method of claim 11, wherein the plant extract is an extract of Camellia spec., Hibiscus spec., Malva spec., Vitis spec., Daucus spec., Commiphora spec., Simmondsia spec., or Calendula spec. or mixtures thereof.

15. The method of claim 11, wherein the plant extract is an extract of Camellia sinensis, Hibiscus sabdariffa, Malva sylvestris, Vitis viticola, Daucus carota, Commiphora myrrha, Simmondsia chinensis or Calendula officinalis or mixtures thereof.

16. The method of claim 1, wherein the substance having a probiotic action is a glycerol monoalkyl ether.

17. The method of claim 16, wherein the glycerol monoalkyl ether is 1-(2-ethylhexyl) glycerol ether.

18. The method of claim 1, wherein the substance having a probiotic action is an ester of an organic acid.

19. The method of claim 1, wherein the ester is isopropyl myristate or ethyl myristate.

20. A cosmetic or pharmaceutical composition comprising a substance having a probiotic effect on the skin.

21. The composition of claim 20, wherein the substance having a probiotic effect is a plant extract.

22. The composition of claim 20, wherein the plant extract is a tea extract, an extract from the group of Vitaceae, Asteraceae, Apiaceae or Buxaceae or mixtures thereof.

23. The composition of claim 22, wherein the tea extract is Theaceae or Malvaceae.

24. The composition of claim 21, wherein the plant extract is an extract of Camellia spec., Hibiscus spec., Malva spec., Vitis spec., Daucus spec., Commiphora spec., Simmondsia spec. or Calendula spec. or mixtures thereof.

25. The composition of claim 21, wherein the plant extract is an extract of Camellia sinensis, Hibiscus sabdariffa, Malva sylvestris, Vitis viticola, Daucus carota, Commiphora myrrha, Simmondsia chinensis or Calendula officinalis or mixtures thereof.

26. The composition of claim 20, wherein the substance having a probiotic effect is a glycerol monoalkyl ether.

27. The composition of claim 26, wherein the glycerol monoalkyl ether is 1-(2-ethylhexyl) glycerol ether.

28. The composition of claim 26, wherein the substance having a probiotic action is an ester of an organic acid.

29. The composition of claim 28, wherein the ester is isopropyl myristate or ethyl myristate.

30. The composition of claim 20, further comprising a substance selected from the group consisting of organic, mineral and modified mineral photoprotective filters, vitamins, provitamins, vitamin precursors from the vitamin B group or derivatives thereof, derivatives of 2-furanone, panthenol, pantolactone, nicotinamide and biotin, additional plant extracts, MMP-1-inhibiting substances, esters of retinol with a C₂₋₁₈-carboxylic acid, enzyme inhibitors, stabilizers, deodorant active ingredients, oils and waxes.

31. The composition of claim 20, wherein the composition is a topical skin-treatment composition.

32. The composition of claim 20, wherein the composition is a soap, a lotion, a powder, a syndet, a form, a stick, an emulsion, a spray, a cream, a gel, a shampoo or a plaster.

33. The composition of claim 20, wherein the composition is a deodorant or an antiperspirant.

34. A method for identifying substances with a probiotic action in the axillary area, comprising the steps of:
a) obtaining a sample of axillary body fluid from a subject having a strong or weak body odor;
b) culturing the axillary body fluid to identify odor-forming microbes;
c) screening a substance library for substances which inhibit the growth of the odor-forming microbes.

35. The product of the process of claim 34.

36. The process of claim 34, wherein the library comprises substances which promote the growth and/or the physiological activity of odor-neutral Staphylococci and/or at the same time inhibit the growth and/or the physiological activity of odor-forming Staphylococci and/or of Gram-positive anaerobic cocci.
